(19) United States
(12) Patent Application Publication
(10) Pub. No.: US 2006/0134670 A1
(43) Pub. Date: Jun. 22, 2006

(54) Enabling tools to identify ligands for hormone nuclear receptors

(76) Inventor: Fabrice Piu, San Diego, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 11/283,329

(22) Filed: Nov. 18, 2005

Related U.S. Application Data
(60) Provisional application No. 60/629,811, filed on Nov. 19, 2004.

(51) Int. Cl. C12Q 1/68; G01N 33/53

(52) U.S. Cl. 435/6; 435/7.1

(57) ABSTRACT

A method and kit developed to identify substances that act as ligands, corepressors, coactivators, agonist and antagonists for cloned nuclear hormone receptors, as well as a test kit for use in the methods is provided herein. More specifically, the method involves expressing a nuclear hormone receptor, receptor heterodimer, and/or receptor homodimer, DNA encoding one or more signaling molecules and DNA encoding a marker, incubating the cells with a test substance, and identifying whether the test substance interacts with the receptor quantitatively or qualitatively by identifying the amount of marker and/or the proliferation of the cells.

### FIELD OF INVENTION

The present invention relates to methods developed to identify substances that act as ligands or helper proteins (agonists, antagonists, inverse agonists and selective modulators) for cloned nuclear hormone receptors, as well as a test kit for use in the methods.

### Incorporation by Reference of Material Submitted on a Compact Disc

The present application is being filed along with duplicate copies of a CD-ROM marked “Copy 1” and “Copy 2” containing a Sequence Listing in electronic format. The duplicate copies of the CD-ROM each contain a file entitled ACADIA.043A.txt created on Nov. 18, 2005 which is 177,000 Bytes in size. The information on these duplicate CD-ROMs is incorporated herein by reference in its entirety.

### BACKGROUND OF THE INVENTION

An important focus of the pharmaceutical drug discovery process is the identification of surrogate ligands for receptor proteins. In the case of nuclear hormone receptors, most of the receptors have been cloned (especially for those receptors present in humans) and pharmacologically characterized. Now, the pharmaceutical industry is attempting to isolate substances that act as ligands for nuclear hormone receptors by screening vast libraries of chemical entities (natural and artificial). Unfortunately, the available methods and technologies create significant constraints that hamper the ability to efficiently screen these libraries against so many targets.

Nuclear hormone receptors, more commonly referred to as nuclear receptors, define a family of ligand activated transcription factors (Tenbaum et al, Int J Biochem Cell Biol, 29:1325-41 (1997); Willson et al, Mol Endocrinol, 16:1135-44 (2002)). Structurally, they are characterized by the presence of modular domains: a zinc-finger DNA binding domain, a ligand binding domain and two transcriptional activation domains AF-1 and AF-2, ligand-independent and ligand-dependent, respectively. Depending upon the nuclear receptor, monomers or dimers (homodimers or heterodimers with the RXR nuclear receptor) constitute the functional effectors. This gene family regulates a wide variety of physiological functions and has thus a broad therapeutic potential ranging from metabolic, endocrinological diseases to neurological disorders, to cancer.

Nuclear receptors operate by recruiting an array of auxiliary polypeptides, denoted corepressors and coactivators, and it is these auxiliary proteins that mediate the molecular events that result in transcriptional repression or activation. For most nuclear receptors, this recruitment event is initiated upon the binding of the nuclear receptor to a ligand. It can be envisioned that certain ligands can only trigger the recruitment of a particular set of coactivators or corepressors and thus promote very selective effects. Furthermore, phosphorylation/dephosphorylation events can also affect the activity of the nuclear receptor itself and/or the auxiliary proteins. Similarly, it is plausible to assume that certain ligands exclusively responsive to such modifications could be identified. Generally speaking, these selective modulators would be of tremendous interest from a therapeutic standpoint, exhibiting maximized therapeutic value and minimum adverse effects.

The first step in the characterization of ligand interaction with a cloned receptor is to express the receptor in a ligand sensitive form. While a few receptors can be expressed in easily manipulated model systems such as yeast and E. coli, the interactions of ligands with most receptors are influenced by post-translational modifications that are only present in mammalian cells. Moreover, many of these receptors require mammalian proteins to accurately transduce their biological effects. Thus for wide applicability, an assay system is best when it is based on cloned receptors expressed in a mammalian system.

Historically, the ability of ligands to interact with nuclear receptors has been evaluated by competition with a radiolabeled ligand for a binding site on the receptor. Such assays are popular because they involve relatively few steps. However binding assays have many limitations: (i) for many technical reasons, binding assays are performed in non-physiological conditions which can influence receptor pharmacology; (ii) agonists and antagonists cannot be reliably discriminated; (iii) only binding sites for which radiolabeled ligands are available can be studied; (iv) binding assays are not easily applicable to orphan receptors for which ligands haven't yet been identified; (v) purchase, handling and disposal of radioisotopes are major expenses.

To reliably discriminate between agonist and antagonist ligands, a functional response of the nuclear receptor is measured. Responses to agonist activation of receptors are commonly measured as altered activity of various endogenous cellular proteins. Examples include measurement of interaction between nuclear receptors and coactivators, of transcriptional activation of the nuclear receptor. The former has led to the development of fluorescence based assays such as Fluorescence Polarization (FP) and Time Resolved Fluorescence Resonance Energy Transfer (TR-FRET). The latter involves conveniently assayed marker proteins that can be controlled by the transcriptional activation of the nuclear receptor. This approach resulted in convenient assays of receptors that function as transcription factors.

A theoretical limitation inherent to all of the above methods is their inability to assay a given ligand against more than a few receptors at the same time. For example, FP and TR-FRET assays rely on specific interactions between nuclear receptors and specific co-activators. Also, these assays have very poor pharmacological characteristics. More generally, because of their limited dynamic range, incompatible assay conditions, and the fact that many receptors cannot be distinguished from one another based upon their functional responses, these are not amenable to multiplexed assays.

### SUMMARY OF THE INVENTION

Aspects of the invention relate to methods for enabling or improving assays of nuclear receptor function, by performing the assays in a cell which expresses one or more helper proteins and one or more nuclear receptors. In some embodiments, a nucleic acid encoding one or more nuclear receptors or a nucleic acid encoding one or more helper proteins can be introduced into the cell. In other embodiments, a nucleic acid encoding one or more nuclear receptors and a nucleic acid encoding one or more helper proteins can be introduced into the cell. In some embodiments, the one or more nuclear receptors and one or more helper proteins are encoded by different nucleic acids, whereas in other embodiments, the nuclear receptor(s) and helper protein(s) are encoded by the same nucleic acid. In some embodiments, the cells can be contacted with a substance and it can be determined whether the substance modulates the activity of the receptor positively or negatively, thereby indicating whether the substance is a ligand for a nuclear receptor.

In some embodiments, assays of nuclear receptor function preformed in a cell which expresses one or more helper proteins and one or more nuclear receptors, can also include a cellular parameter to detect or validate the function of nuclear receptors whose functions or abilities to function are unknown. Some embodiments relate to assays of nuclear receptor function preformed in a cell which expresses one or more helper proteins and one or more nuclear receptors, also including a step of evaluating the signal transduction properties of said one or more nuclear receptors whose functions or abilities to function are unknown and thereby optimizing assays for those receptors.

In some embodiments of any of the methods described above, wherein the one or more nuclear receptors is encoded by a nucleic acid, at least one nucleotide sequence can be selected from the group including SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, and 143. In some embodiments of any of the methods described above, the one or more nuclear receptors can be encoded by a nucleic acid having at least 70% identity to a nucleotide sequence selected from the group including: SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, and 143.

In some embodiments of any of the methods described above, the helper proteins can enable a response to receptor activation that the receptor does not normally produce. In other embodiments of any of the methods described above, the helper proteins can amplify responses that the receptor normally produces. In still other embodiments, the helper proteins can amplify responses that the receptor does not normally produce but that are enabled by other helper proteins. In yet other embodiments, the helper proteins can block receptor responses that interfere with detection of the primary functional response of the receptor.

In some embodiments of any of the methods described above, the helper protein can a co-activator encoded by a nucleic acid that includes a nucleotide sequence selected from the group including: SEQ ID NOs: 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, and 241, or a nucleic acid having at least 70% sequence identity to a nucleotide sequence selected from the group including: SEQ ID NOs: 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, and 241.

In some embodiments of any of the methods described above, the helper protein can be a co-repressor encoded by a nucleic acid comprising a nucleotide sequence selected from the group consisting of SEQ ID NOs: 195, 197, 199, 201, and 203, or a nucleic acid having at least 70% sequence identity to a nucleotide sequence selected from the group including: SEQ ID NOs: 195, 197, 199, 201, and 203.

In some embodiments of any of the methods described above, the helper protein is a kinase encoded by a nucleic acid comprising a nucleotide sequence selected from the group including: SEQ ID NOs: 205, 207, 209, 211, 213, 215, 217, 219, 221, 223, 225, 227, 229, 231, 233, 235, 237, 239, and 241, or a nucleic acid having at least 70% sequence identity to a nucleotide sequence selected from the group including: SEQ ID NOs: 205, 207, 209, 211, 213, 215, 217, 219, 221, 223, 225, 227, 229, 231, 233, 235, 237, 239, and 241.

In other embodiments of any of the methods described above, the helper proteins can be chimeras between two or more proteins that redirect signal transduction pathways, linking domains that receive regulatory or signal inputs to domains that provide effector or signal outputs.

In embodiments of any of the methods described above, the helper proteins can be naturally occurring proteins that are not normally expressed in the cell used for the functional assay. In other embodiments, the helper proteins can bee naturally occurring proteins that are normally expressed in the cell used for the functional assay that are overexpressed.

In some embodiments of any of the methods described above, the helper proteins can be truncated or mutated versions of naturally occurring proteins that are not normally expressed in the cell used for the functional assay. For example, in some embodiments, the helper proteins can be truncated or mutated versions of naturally occurring proteins that are normally expressed in the cell used for the functional assay that are overexpressed.

In some embodiments of any of the methods described above, the helper proteins can be mixtures of 2 or more proteins, chimeras, mutant proteins, or truncated proteins which, when co-expressed, enable or improve detection of functional responses to nuclear hormone receptors. For example, in some embodiments, the helper proteins can be other naturally and non-naturally occurring receptors that help the receptor being functionally assayed to signal better. In other embodiments, the helper proteins can be other naturally and non-naturally occurring receptors that help the expression and formation of the receptor being functionally assayed. In other embodiments, the helper proteins can be other naturally and non-naturally occurring receptors that help the receptor being functionally assayed to respond more sensitively to ligands

Other embodiments relate to a method of assessing the effect of a candidate compound on the activity of a nuclear receptor comprising obtaining a cell expressing a nuclear receptor and a helper protein, wherein at least one of the nuclear receptor and the helper protein is expressed from a nucleic acid which has been introduced into the cell, contacting the cell with the candidate compound, and determining whether the candidate compound influences the activity of the nuclear hormone receptor. In some embodiments a method for identifying ligands for cloned nuclear receptors is provided. In some embodiments, a method for identifying ligands by simultaneous screening of compounds for activity at multiple cloned receptors is provided. In some embodiments, a method for measuring ligand concentration by activity at the nuclear receptors is provided. In other embodiments, a method for employing recombinant signaling molecules to facilitate assay of ligands for nuclear receptors is provided. In other embodiments, a method to identify DNAs encoding nuclear receptors for ligands is provided. In other embodiments a method of identifying mutant forms of nuclear receptors that have altered ligand dependence is provided.

One embodiment is a method of detecting a substance which is a ligand of a nuclear hormone receptor, the method comprising expressing one or more nuclear hormone receptors and one or more helper proteins in a cell, contacting the cell with a candidate substance, and determining whether said candidate substance influences the activity of the nuclear receptor. In one aspect of the embodiment, the DNA encoding the nuclear receptor comprises a sequence selected from the group consisting of SEQ ID NOs: 1, 3, 5, 7, 9, 11, 13, 15, 17, 19, 21, 23, 25, 27, 29, 31, 33, 35, 37, 39, 41, 43, 45, 47,49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, and 143. In one aspect, the helper protein is a coactivator and the DNA encoding the coactivator comprises a sequence selected from the group consisting of SEQ ID NOs.: 145, 147, 149, 151, 153, 155, 157, 159, 161, 163, 165, 167, 169, 171, 173, 175, 177, 179, 181, 183, 185, 187, 189, 191, and 193. In yet a further aspect, the ligand is an agonist, antagonist, inverse agonist or selective modulator. In a further embodiment, the helper protein is a corepressor and the DNA encoding the corepressor comprises a sequence selected from the group consisting of SEQ ID NOs: 195, 197, 199, 201, and 203. In yet a further embodiment, the ligand is an agonist, antagonist, inverse agonist or selective modulator. In a further embodiment, the helper protein is a kinase and DNA encoding the kinase comprises a sequence selected from the group consisting of SEQ ID NOs: 205, 207, 209, 211, 213, 215, 217, 219, 221, 223, 225, 227, 229, 231, 233, 235, 237, 239, and 241. In yet a further embodiment, the helper protein is a ligand and the ligand is an agonist, antagonist, inverse agonist or selective modulator. In a further embodiment the helper proteins is a signaling molecules and the signaling molecule is defined as any polypeptide which directly or indirectly modulates the activity of nuclear hormone receptors. In yet a further aspect, the ligand is an agonist, antagonist, inverse agonist or selective modulator.

A further embodiment is a test kit for detecting a substance capable of acting as a ligand for a nuclear hormone receptor, the kit comprising: (a) expressing a nuclear receptor and one or more coactivators, or (b) expressing a nuclear receptor and one or more corepressors, or (c) expressing a nuclear receptor and one or more kinases, or (d) expressing a nuclear receptor and one or more signaling molecules. In one aspect of the embodiment, the ligand is an agonist, an antagonist, an inverse agonist or a selective modulator.

A further embodiment of the invention is a method for detecting a mutant form of a receptor or mutant form of a helper protein associated with the receptor, which mutant form will affect the response to a ligand on said cloned nuclear receptor present as one or more of the following combinations: (a) expressing a nuclear receptor and one or more coactivators, or (b) expressing a nuclear receptor and one or more corepressors, or (c) expressing a nuclear receptor and one or more kinases, or (d) expressing a nuclear receptor and one or more signaling molecules. One embodiment of the invention is a method for detecting a mutant form of a receptor or mutant form of a signal transducing protein associated with the receptor, which mutant form will affect the response to a ligand on said cloned nuclear receptor present as one or more of the following combinations: (a) expressing a nuclear receptor and one or more coactivators, or (b) expressing a nuclear receptor and one or more corepressors, or (c) expressing a nuclear receptor and one or more kinases, or (d) expressing a nuclear receptor and one or more signaling molecules.

A further embodiment of the invention is a method of detecting a substance which is a ligand of a nuclear hormone receptor, the method comprising one of the following: expressing a nuclear hormone receptor and one or more coactivators, expressing a nuclear hormone receptor and one or more corepressors, expressing a nuclear hormone receptor and one or more kinases, expressing a nuclear hormone receptor and one or more signaling molecules.

One embodiment is a method for enabling or improving assays of nuclear receptor function by performing assays in a cell which expresses one or more helper proteins and one or more nuclear receptors. In one embodiment, a nucleic acid encoding the one or more nuclear receptors or a nucleic acid encoding the one or more helper proteins is introduced into the cell.

In a further embodiment, a nucleic acid encoding the one or more nuclear receptors and a nucleic acid encoding the one or more helper proteins has been introduced into the cell. The one or more nuclear receptors and the one or more helper proteins can be encoded by different nucleic acids or can be encoded by the same nucleic acid. In a further embodiment, the method can also include contacting the cell with a substance and determining whether the substance is a ligand for a nuclear receptor. The ligand can modulate the activity of the nuclear receptor positively or negatively. The method can also involve evaluating a cellular parameter to detect or validate the function of nuclear receptors whose functions or abilities to function are unknown. Alternatively, or in addition, the method can involve evaluating the signal transduction properties of the one or more nuclear receptors whose functions or abilities to function are unknown and thereby optimizing assays for those receptors. The one or more nuclear receptors can be encoded by a nucleic acid including at least one nucleotide sequence selected from the group consisting of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, and 143 or at least 70% identical. The helper proteins can enable a response to receptor activation that the receptor does not normally produce and/or can amplify responses that the receptor normally produces. In one embodiment, the helper proteins amplify responses that the receptor does not normally produce but that are enabled by other helper proteins or alternatively, they block receptor responses that interfere with detection of the primary functional response of the receptor. The helper protein can be a co-activator encoded by a nucleic acid including a nucleotide sequence selected from the group consisting of SEQ ID NOs: 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, and 241 or 70% identical. The helper protein can be a co-repressor encoded by a nucleic acid including a nucleotide sequence selected from the group consisting of SEQ ID NOs: 195, 197, 199, 201, and 203 or 70% identical. The helper protein can be a kinase encoded by a nucleic acid including a nucleotide sequence selected from the group consisting of SEQ ID NOs: 205, 207, 209, 211, 213, 215, 217, 219, 221, 223, 225, 227, 229, 231, 233, 235, 237, 239, and 241 or 70% identical. In further embodiments, the helper proteins are chimeras between two or more proteins that redirect signal transduction pathways, linking domains that receive regulatory or signal inputs to domains that provide effector or signal outputs. The helper proteins can be naturally occurring proteins that are not normally expressed in the cell used for the functional assay, naturally occurring proteins that are normally expressed in the cell used for the functional assay that are overexpressed, truncated or mutated versions of naturally occurring proteins that are not normally expressed in the cell used for the functional assay, or truncated or mutated versions of naturally occurring proteins that are normally expressed in the cell used for the functional assay that are overexpressed or mixtures thereof. In one embodiment, the helper proteins are other naturally and non-naturally occurring receptors that help the receptor being functionally assayed to signal better, other naturally and non-naturally occurring receptors that help the expression and formation of the receptor being functionally assayed, or other naturally and non-naturally occurring receptors that help the receptor being functionally assayed to respond more sensitively to ligands.

One embodiment is a method of assessing the effect of a candidate compound on the activity of a nuclear receptor by obtaining a cell expressing one or more nuclear receptors and one or more helper proteins, wherein at least one of the nuclear receptor and the helper protein is expressed from a nucleic acid which has been introduced into the cell; contacting the cell with the candidate compound; and determining whether the candidate compound influences the activity of the nuclear hormone receptor.

In one embodiment, both the one or more nuclear receptor and the one or more helper protein are expressed from a nucleic acid which has been introduced into the cell. Alternatively, the one or more nuclear receptor and the one or more helper protein are expressed from the same nucleic acid which has been introduced into the cell. Alternatively, the one or more nuclear receptor is expressed from a first nucleic acid which has been introduced into the cell and the helper protein is expressed from a second nucleic acid which has been introduced into the cell.

In one embodiment, the determining step comprises comparing the activity of the nuclear hormone receptor in a first cell which expresses the nuclear receptor and the helper protein and which has been contacted with the candidate compound to the activity of the nuclear receptor in a second cell which expresses the nuclear receptor and the helper protein and which has not been contacted with the candidate compound, wherein the candidate compound is determined to influence the activity of the nuclear receptor if the activity of the nuclear receptor in the first cell is significantly different from the activity of the nuclear receptor in the second cell.

In one embodiment, the one or more nuclear receptors is encoded by a nucleic acid selected from the group consisting of SEQ ID NOs.: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, and 143 and the one or more helper proteins is encoded by a nucleic acid selected from the group consisting of 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, and 241 or sequences 70% identical to the above sequences.

In a further embodiment, the one or more nuclear receptors comprises an amino acid sequence selected from the group consisting of SEQ ID NOs: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, and 144 and wherein the one or more helper proteins comprises an amino acid sequence selected from the group consisting of SEQ ID NOs: 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, and 242 or an amino acid sequence having at least 70% identity to any of the above sequences.

In one embodiment, the combination of the nucleic acid sequence encoding the one or more nuclear receptors and the one or more helper proteins are selected from the group consisting of: SEQ ID NO: 5 and SEQ ID NOs: 161 or 213; SEQ ID NO: 9 and SEQ ID NOs: 145, 147, 161, or 213; SEQ ID NOs: 21, 23, 25, or 27 and SEQ ID NOs: 145, 147, or 213; SEQ ID NO: 49 and SEQ ID NOs: 145, 147, 161, or 213; SEQ ID NO: 45 and SEQ ID NOs: 145 or 213; SEQ ID NO: 51 and SEQ ID NOs: 145 or 147; SEQ ID NOs: 53, 55, or 57 and SEQ ID NOs: 145 or 147; SEQ ID NO: 69 and SEQ ID NOs: 161 or 213; SEQ ID NO: 93 and SEQ ID NOs: 145, 147, 161, or 213; SEQ ID NO: 107 and SEQ ID NO: 161; SEQ ID NO: 103 and SEQ ID NOs: 145, 147, or 161; SEQ ID NO: 101 and SEQ ID NOs: 145 or 147; SEQ ID NO: 143 and SEQ ID NOs: 145, 147 or 161; SEQ ID NO: 29 and SEQ ID NO: 213; SEQ ID NO: 43 and SEQ ID NO: 161; SEQ ID NO: 61 and SEQ ID NOs: 145, 147, 161 or 213; SEQ ID NO: 75 and SEQ ID NOs: 145 or 147; SEQ ID NO: 79 and SEQ ID NO: 161; SEQ ID NO: 87 and SEQ ID NO: 145, 147, or 161; SEQ ID NO: 89 and SEQ ID NOs: 145, 147 or 161; SEQ ID NO: 99 and SEQ ID NO:161; SEQ ID NOs: 123, 125, or 127 and SEQ ID NOs: 145, 147, 161, or 213; and SEQ ID NO: 135 and SEQ ID NOs: 145, 147 or 213.

In a further embodiment, the combination of the amino acid sequence of the one or more nuclear receptors and the one or more helper proteins are selected from the group consisting of: SEQ ID NO: 6 and SEQ ID NOs: 162 or 214; SEQ ID NO: 10 and SEQ ID NOs: 146, 148, 162, or 214; SEQ ID NOs: 22, 24, 26 or 28 and SEQ ID NOs: 146, 148, or 214; SEQ ID NO: 50 and SEQ ID NOs: 146, 148, 162, or 214; SEQ ID NO: 46 and SEQ ID NOs: 146, 148 or 214; SEQ ID NO: 52 and SEQ ID NOs: 146 or 148; SEQ ID NOs: 54, 56, or 58 and SEQ ID NOs: 146 or 148; SEQ ID NO: 70 and SEQ ID NOs: 162 or 214; SEQ ID NO: 94 and SEQ ID NOs: 146, 148, 162, or 214; SEQ ID NO: 108 and SEQ ID NO: 162; SEQ ID NO: 104 and SEQ ID NOs: 146, 148, or 162; SEQ ID NO: 102 and SEQ ID NOs: 146 or 148; SEQ ID NO: 144 and SEQ ID NOs: 146, 148, or 162; SEQ ID NO: 30 and SEQ ID NO: 214; SEQ ID NO: 44 and SEQ ID NO: 162; SEQ ID NO: 62 and SEQ ID NOs: 146, 148, 162, or 214; SEQ ID NO: 76 and SEQ ID NOs: 146 or 148; SEQ ID NO: 80 and SEQ ID NO: 162; SEQ ID NO: 89 and SEQ ID NO: 146, 148, or 162; SEQ ID NO: 90 and SEQ ID NOs: 146, 148, or 162; SEQ ID NO: 100 and SEQ ID NO:162; SEQ ID NOs: 124, 126, or 128 and SEQ ID NOs: 146, 148, 162, or 214; and SEQ ID NO: 136 and SEQ ID NOs: 146, 148 or 214.

In a further embodiment the combination of the nuclear receptor expressed by the cell and the helper protein expressed by the cell are selected from the group consisting of: TR beta and DRIP 205 or ERK2; RAR beta and SRC1, DRIP205 or ERK2; PPAR gamma/RXR and SRC1 or ERK2; FXR/RXR and SRC1, DRIP205 or ERK2; LXR beta/RXR and SRC1 or ERK2; VDR/RXR and SRC1; PXR and SRC1; RXR alpha and DRIP205 or ERK2; ER beta and SRC1, DRIP205 or ERK2; AR and DRIP205; MR and SRC1 or DRIP205; GR and SRC1; SHP and SRC1 or ERK2; RevERb alpha and ERK2; ROR gamma and DRIP205; HNF4 alpha and SRC1, DRIP205 or ERK2; TR2 alpha and SRC1; TLX and ERK2; COUP-TF beta and SRC1 or DRIP205; EAR2 and SRC1, DRIP205 or ERK2; ERR gamma and ERK2; NOR-1 and SRC1, DRIP205 or ERK2; and GCNF and SRC1 or ERK2.

In one embodiment the determining step involves determining whether the compound influences the activity of the one or more nuclear receptors by evaluating a cellular parameter selected from the group consisting of morphology, phosphorylation, differentiation, apoptosis, process formation, motility, gene expression, expression of a cellular receptor, and a phenotypic change. In a further embodiment, the method includes introducing a nucleic acid including a promoter from which the level of transcription is responsive to activation of the nuclear receptor into the cell, the promoter being operably linked to a nucleic acid encoding a detectable product and determining whether the candidate compound influences the activity of the nuclear receptor by measuring the amount of the detectable product.

A further embodiment is a method of identifying interaction between a nuclear receptor and one or more helper proteins, the method by: co-transfecting a first cell culture with DNA encoding a nuclear receptor and DNA encoding a marker of cell amplification, along with DNA encoding the one or more helper proteins; incubating the first cell culture for a period of time sufficient to permit cell amplification of the transfected cells; co-transfecting a second cell culture with DNA encoding a nuclear receptor and DNA encoding a marker of cell amplification; incubating the second cell culture for a period of time sufficient to permit cell amplification of the transfected cells; and determining whether the one or more helper proteins interact with the nuclear receptor by comparing the level of amplification of transfected cells expressing the nuclear receptor and the one or more helper proteins to the level of amplification of cells which were transfected with DNA encoding the nuclear receptor but which were not transfected with DNA encoding the one or more helper proteins.

In one embodiment, the helper protein is selected from the group consisting of SEQ ID NOs: 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, and 242 or having 70% identify thereto.

In a further embodiment, the DNA encoding the nuclear receptor comprises a sequence selected from the group consisting of SEQ ID NOs. 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, and 143 or having 70% identify thereto.

In a further embodiment, the DNA encoding the nuclear receptor encodes a polypeptide including a sequence selected from the group consisting of SEQ ID NOs.: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, and 144 or having 70% identify thereto.

In a further embodiment, the DNA encoding the one or more helper proteins comprises a sequence selected from the group consisting of SEQ ID NOs.: 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, and 241 or having 70% identity thereto.

In a further embodiment, the DNA encoding the one or more helper proteins encodes a polypeptide including a sequence selected from the group consisting of SEQ ID NOs.: 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, and 242 or having 70% identify thereto.

In a further embodiment, the DNA encoding the nuclear receptor and the DNA encoding the marker of cell amplification can be on the same vector, or on separate vectors.

In a further embodiment, the step of incubating the first cell culture for a period of time sufficient to permit cell amplification of the transfected cells comprises contacting the first cell culture with a ligand which binds to the nuclear receptor and wherein the step of incubating the second cell culture for a period of time sufficient to permit cell amplification of the transfected cells comprises contacting the second cell culture with a ligand which binds to the nuclear receptor. The ligand can be an agonist or an antagonist.

One embodiment is a method of identifying interaction between a nuclear receptor and one or more helper proteins, the method by: co-transfecting a first cell culture with DNA encoding a nuclear receptor and DNA encoding a marker of cell amplification, along with DNA encoding one or more helper proteins; incubating the cell culture with varying concentrations of a ligand which is an agonist or antagonist for the nuclear receptor for a period of time sufficient to permit cell amplification of the transfected cells in the first cell culture; co-transfecting a second cell culture with DNA encoding a nuclear receptor and DNA encoding a marker of cell amplification; incubating the cell culture with varying concentrations of a ligand which is an agonist or antagonist for the nuclear receptor for a period of time sufficient to permit cell amplification of the transfected cells in the second cell culture; determining whether the one or more helper proteins interact with the nuclear receptor by comparing the level of amplification of transfected cells expressing the nuclear receptor and the one or more helper proteins to the level of amplification of cells which were transfected with DNA encoding the nuclear receptor but which were not transfected with DNA encoding the one or more helper proteins. The one or more helper proteins can be coactivators, corepressors, kinases, signaling molecules, or at least two of the above.

In one embodiment, the DNA encoding the nuclear receptor comprises a sequence selected from the group consisting of SEQ ID NOs. 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, and 143. In a further embodiment, the DNA encoding the nuclear receptor encodes a polypeptide including a sequence selected from the group consisting of SEQ ID NOs.: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, and 144.

In one embodiment, the DNA encoding the one or more helper proteins comprises a sequence selected from the group consisting of SEQ ID NOs.: 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, and 241. In a further embodiment, the DNA encoding the one or more helper proteins encodes a polypeptide including a sequence selected from the group consisting of SEQ ID NOs.: 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, and 242. The DNA encoding the nuclear receptor and the DNA encoding the marker of cell amplification can be on the same vector or on separate vectors.

A further embodiment is a method of identifying a substance which is a ligand of a nuclear receptor, the method by: incubating a cell culture which comprises a mixture of cells transfected with DNA encoding a nuclear receptor, DNA encoding a marker of cell amplification and DNA encoding one or more helper proteins and untransfected cells, with a test substance which is a potential agonist or antagonist for the nuclear receptor for a period of time sufficient to permit cell amplification of the transfected cells; and determining any increase or decrease in cell amplification by measuring the level of the marker in the transfected cells.

A further embodiment is a method of identifying a substance which is a selective modulator of a particular combination of a nuclear receptor and one or more helper proteins, the method by: co-transfecting a first cell culture including cells of a first cell type with DNA encoding a nuclear receptor and DNA encoding a marker of cell amplification, along with DNA encoding the one or more helper proteins; incubating the first cell culture with a test substance; determining whether the test substance increases or decreases amplification of the transfected cells of the first cell type relative to untransfected cells of the first cell type; co-transfecting a second cell culture including cells of a second cell type with DNA encoding the nuclear receptor and DNA encoding the marker of cell amplification, along with DNA encoding the one or more helper proteins; incubating the second cell culture with the test substance; determining whether the test substance increases or decreases amplification of the transfected cells of the second cell type relative to untransfected cells of the second cell type; wherein the test substance is a selective modulator of the nuclear receptor if the effects of the test substance on the first cell type are opposite to the effects of the test substance on the second cell type.

A further embodiment is a method of identifying a substance which is a selective modulator of a particular combination of a nuclear receptor and one or more helper proteins, the method by: co-transfecting a first cell culture with DNA encoding a nuclear receptor and DNA encoding a marker of cell amplification, along with DNA encoding one or more first helper proteins; incubating the first cell culture with a test substance; determining whether the test substance increases or decreases amplification of the transfected cells in the first cell culture relative to untransfected cells; co-transfecting a second cell culture with DNA encoding the nuclear receptor and DNA encoding a marker of cell amplification, along with DNA encoding a second one or more helper proteins, wherein the second one or more helper proteins are distinct from the first one or more helper proteins; incubating the second cell culture with the test substance; determining whether the test substance increases or decreases amplification of the transfected cells in the second cell culture relative to untransfected cells; wherein the test substance is a selective modulator of the nuclear receptor if the effects of the test substance on the first cell culture are opposite to the effects of the test substance on the second cell culture.

A further embodiment is a method of identifying a substance which is a selective modulator of a nuclear receptor by: co-transfecting a first cell culture including cells of a first cell type with DNA encoding a nuclear receptor and DNA encoding a marker of cell amplification; incubating the first cell culture with a test substance; determining whether the test substance increases or decreases amplification of the transfected cells of the first cell type relative to untransfected cells of the first cell type; co-transfecting a second cell culture including cells of a second cell type with DNA encoding the nuclear receptor and DNA encoding the marker of cell amplification; incubating the second cell culture with the test substance; determining whether the test substance increases or decreases amplification of the transfected cells of the second cell type relative to untransfected cells of the second cell type; wherein the test substance is a selective modulator of the nuclear receptor if the effects of the test substance on the first cell type are opposite to the effects of the test substance on the second cell type.

A further embodiment is a method of identifying interaction between two nuclear receptors by: co-transfecting a first cell culture with DNA encoding a first nuclear receptor, DNA encoding a second nuclear receptor and DNA encoding a marker of cell amplification; incubating the first cell culture for a period of time sufficient to permit cell amplification of the transfected cells in the first cell culture; co-transfecting a second cell culture with DNA encoding a marker of cell amplification and either DNA encoding the first nuclear receptor or DNA encoding the second nuclear receptor; incubating the second cell culture for a period of time sufficient to permit cell amplification of the transfected cells in the second cell culture; and determining whether the nuclear receptors interact with one another by comparing the level of amplification of transfected cells expressing both the nuclear receptors to the level of amplification of cells which were transfected with DNA encoding the marker of cell amplification and either DNA encoding the first nuclear receptor or DNA encoding the second nuclear receptor.

A further embodiment is a method of identifying interaction between two nuclear receptors and one or more helper proteins by: co-transfecting a first cell culture with DNA encoding a first nuclear receptor, DNA encoding a second nuclear receptor, DNA encoding one or more helper proteins and DNA encoding a marker of cell amplification; incubating the first cell culture for a period of time sufficient to permit cell amplification of the transfected cells; co-transfecting a second cell culture with DNA encoding one of the nuclear receptors, DNA encoding the one or more helper proteins and DNA encoding the marker of cell amplification or with DNA encoding both nuclear receptors and DNA encoding the marker of cell amplification; and determining whether the two nuclear receptors and one or more helper proteins interact with one another by comparing the level of amplification of transfected cells in the first cell culture to the level of amplification of transfected cells in the second cell culture.

One embodiment is a method of detecting a substance which is a ligand of two nuclear receptors by: incubating a cell culture which comprises a mixture of cells transfected with DNA encoding a first nuclear receptor, DNA encoding a second nuclear receptor, and DNA encoding a marker of cell amplification with a test substance which is a potential agonist or antagonist for the nuclear receptor for a period of time sufficient to permit cell amplification of the transfected cells; and determining any increase or decrease in cell amplification by measuring the level of the marker of cell amplification in the transfected cells.

A further embodiment is a method of detecting a substance which is a selective modulator of a particular combination of two nuclear receptors and one or more helper proteins by: co-transfecting a first cell culture having cells of a first cell type with DNA encoding a first nuclear receptor, DNA encoding a second nuclear receptor, DNA encoding one or more helper proteins, and DNA encoding a marker of cell amplification; incubating the first cell culture with a test substance; determining whether the test substance increases or decreases amplification of the transfected cells in the first cell culture relative to untransfected cells in the first cell culture; co-transfecting a second cell culture having cells of a second cell type with DNA encoding the first nuclear receptor, DNA encoding the second nuclear receptor and DNA encoding a marker of cell amplification; incubating the second cell culture with the test substance; and determining whether the test substance increases or decreases amplification of the transfected cells of the second cell type relative to untransfected cells of the second cell type; wherein the test substance is a selective modulator of the nuclear receptor if the effects of the test substance on the first cell type are opposite to the effects of the test substance on the second cell type.

One embodiment is a method of identifying a substance which is a selective modulator of a particular combination of two nuclear receptors and one or more helper proteins by: co-transfecting a first cell culture with DNA encoding a first nuclear receptor, DNA encoding a second nuclear receptor and DNA encoding a marker of cell amplification, along with DNA encoding one or more first helper proteins; incubating the first cell culture with a test substance; determining whether the test substance increases or decreases amplification of the transfected cells in the first cell culture relative to untransfected cells; co-transfecting a second cell culture with DNA encoding the first nuclear receptor, DNA encoding the second nuclear receptor and DNA encoding a marker of cell amplification, along with DNA encoding a second one or more helper proteins, wherein the second one or more helper proteins are distinct from the first one or more helper proteins; incubating the second cell culture with the test substance; determining whether the test substance increases or decreases amplification of the transfected cells in the second cell culture relative to untransfected cells; wherein the test substance is a selective modulator of the nuclear receptor if the effects of the test substance on the first cell culture are opposite to the effects of the test substance on the second cell culture.

### BRIEF DESCRIPTION OF THE DRAWINGS

FIG. 1 is an illustration of an embodiment of the multiple receptor format where agonist induction of the receptors was detected as β-galactosidase activity. Receptor DNAs encoding the glucocorticoid receptor GR alpha and the estrogen receptor ER beta were co-transfected with β-galactosidase marker DNA. Individual cell aliquots were then incubated for varying concentrations of the GR selective ligand dexamethasone and the ER selective ligand estrone.

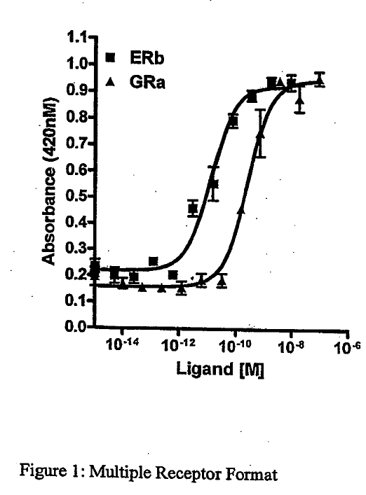

FIG. 2 illustrates how embodiments herein can be used to assess the importance of signaling molecules and particular co-activators in modulating the activity of nuclear hormone receptors. DNAs encoding the PXR (rifampicin receptor) and RXR (retinoic acid receptor) receptors are co-transfected with the β-galactosidase marker DNA in the presence or absence of the co-activators GRIP1 and SRC1 (Glucocorticoid Receptor Interacting Protein 1 and Steroid Receptor Coactivator 1) alone or in combination. Rifampicin is a reference agonist ligand for the PXR/RXR heterodimer.

FIG. 3 is a histogram that illustrates how the present invention can be used to quantify the levels of constitutive activity displayed by nuclear hormone receptors. The Single Receptor Format method was used to transfect increasing concentrations of a nuclear receptor along with the b-galactosidase marker. The data illustrates the relative constitutive activities of the peroxisome PPAR gamma/RXR receptor and the androstane CAR alpha/RXR heterodimer receptor.

FIGS. 4A and 4B illustrate the inverse agonism of nuclear receptors PPARγ/RXR (4B) in the presence of increasing amounts of BRL 49653 and CARα/RXR (4A) in the presence of increasing amounts of Androstenol.

FIG. 5 is a typical pharmacological profile of an agonist response of the retinoid receptor as determined by R-SAT™.

FIG. 6 illustrates how embodiments herein can be used to identify interactions between receptors and signaling molecules. DNAs encoding the indicated RAR receptors were co-transfected with the β-galactosidase marker DNA with or without plasmid DNAs encoding β Arrestin 1 or β Arrestin 2. Cells were contacted with AM-580 and β-galactosidase activity was measured.

FIGS. 7A and 7B are blots of co-immunoprecipitation experiments that demonstrate the interaction between nuclear receptors (RARβ2) and other signaling proteins (Erk, Jnk, P38, and bArr2).

### DETAILED DESCRIPTION OF THE PREFERRED EMBODIMENTS

Disclosed herein are methods of detecting and identifying ligands for nuclear receptors, determining interactions between nuclear receptors and helper proteins, determining interactions between two nuclear receptors, and determining interactions between two nuclear receptors and helper proteins.

Methods are disclosed in which at least one nuclear receptor and at least one helper protein are expressed in a cell. In some embodiments, the cells expressing the at least one nuclear receptor and the at least one helper protein can be used to evaluate the effect of a candidate compound on the activity of the nuclear receptor. In other embodiments, the cells expressing the at least one nuclear receptor and the at least one helper protein can be used to identify helper proteins which interact with a particular nuclear hormone receptor. Other embodiments of the present invention relate to methods for identifying interaction between two nuclear receptors in which the two nuclear receptors are expressed in a cell and their ability to interact with one another is assessed by evaluating a cellular parameter. The interactions being assessed in each of the foregoing methods can be evaluated using any assay capable of detecting such interactions. In preferred embodiments, the interactions being assessed in each of the foregoing methods can be measured by assessing the extent of cellular proliferation in the RSAT assay described in U.S. Pat. Nos. 5,707,798; 5,912,132; and 5,955,281, the disclosures of which are incorporated herein by reference in their entireties). In some embodiments, the effect of the candidate compound on the activity of the receptor can be determined by comparing the activity of the receptor in cells which have been contacted with the candidate compound to the activity of the receptor in cells which have not been contacted with the candidate compound. In some embodiments, the cells are contacted with a known activator or a known inhibitor of the receptor as well as a candidate compound in order to determine the effect of the candidate compound on the action of the activator or inhibitor.

In one embodiment, a method is disclosed of assessing the effect of a candidate compound on the activity of a nuclear receptor by expressing one or more nuclear receptors and one or more helper proteins in a cell, contacting the cell with a candidate compound, and determining whether the candidate compound influences the activity of the receptor. As is true for each of the methods of the present invention, in some aspects of this embodiment at least one or both of the nuclear receptor and the helper protein can be expressed from a nucleic acid which has been introduced into the cell. Any candidate compounds can be used whether they are used to test known compounds or to identify new compounds that interact with the receptor. For example, candidate compounds can be used which include, but are not limited to, small molecules, pharmaceuticals, nucleic acids, peptides, ligands, agonists, and antagonists. In some embodiments, nucleic acids encoding other receptors (including other nuclear receptors),corepressors, co-activators, kinases, and/or signaling molecules are introduced into the cell.

In one embodiment, the method is used for identifying other ‘helper genes’ which encode helper proteins which interact with nuclear hormone receptors and/or for identifying ligands which interact with cloned nuclear hormone receptors in the presence of ‘helper genes’. The molecules which can be identified using the present methods, include but are not limited to, ligands, agonists, antagonists, inverse agonists, and selective modulators for cloned nuclear hormone receptors. The methods can be used for identifying these compounds by simultaneous screening of compounds for activity with respect to single cloned receptors, multiple cloned receptors and mixed receptors that can act as heterodimers. The method can also be used to identify mutant forms of nuclear receptors that have altered ligand dependence, as well as mutant forms of any helper proteins from a group of coactivators, corepressors, kinases or signaling molecules which modulate directly or indirectly the activity of nuclear receptors. The method results in a measurable output which is functionally linked to the assay. The measurable output can be any one known to one of skill in the art which can identify the activity of the ligands and/or ‘helper genes’ and can compare the cells with and without the test compound. In one embodiment, the measurable output is cellular proliferation. In a further embodiment, the measurable output includes, but is not limited to: expression of a gene, phosphorylation, morphology, differentiation, expression of a cellular receptor, apoptosis, any other phenotypic change, or an activity, such as cell motility. In a further embodiment, a reporter gene is expressed from a promoter of interest and the measurable output is analyzed as measured by the expression of the reporter gene.

In one embodiment, the method involves detecting a substance capable of acting as a ligand, agonist, antagonist, inverse agonist or selective modulator by:
(a) culturing cells to express one or more of the following: a nuclear receptor and one or more coactivators, a nuclear receptor and one or more corepressors, a nuclear receptor and one or more kinase, a nuclear receptor and one or more signaling molecules.
(b) incubating the cells with at least one test compound
(c) determining any change in the activity of the nuclear receptor so as to identify a test compound which is a ligand of said nuclear receptor.

In another aspect, a test kit is provided for detecting a substance capable of acting as a ligand, agonist, inverse agonist, antagonist and/or selective modulator, the kit including:
(a) cells expressing at least one of: at least one nuclear receptor and one or more coactivators, or cells expressing a nuclear receptor and one or more corepressors, or cells expressing a nuclear receptor and one or more kinases, or cells expressing a nuclear receptor and one or more signaling molecules.
(b) at least one test compound to incubate with the cells
(c) a method for determining any change in the activity of the nuclear receptor so as to identify a test compound which is a ligand of said nuclear receptor using a measurable output.

This test kit is useful for an embodiment of the present method in which the ability of the test substance (or potentially a large number of test substances) to act as a ligand, agonist, inverse agonist, antagonist and/or selective modulator for a specific receptor is determined by incubation of the test substances with one or more nuclear receptors simultaneously.

The nuclear receptors in each of the methods of the present invention can be any nuclear receptors known to one of skill in the art. The nuclear receptors can be expressed singly in a cell or alternatively multiple nuclear receptors can be expressed within the same cell or group of cells. For example, nuclear receptors which are known to interact, such as heterodimers, can be expressed in the same cells. Alternatively, nuclear receptors which share common ligands or helper proteins can be expressed in the same cell. Alternatively receptors which do not have any known interaction or commonality can be expressed in the same cells. Table 1 provides a non-limiting list of nuclear receptors which can be used in the assays described herein.

The one or more helper genes which express helper proteins can be any helper protein. Helper proteins which are known to interact with specific receptors can be used. Alternatively, helper proteins can be tested to identify whether they interact and modulate a particular receptors. Table 2 provides a non-limiting list of helper proteins which can be used in the assays described herein.

One advantage of using helper proteins in addition to the one or more nuclear receptors is that the activity of the receptor can be enhanced to provide a more easily measured effect. Another advantage is that compounds can be identified which specifically interact with the combination of the nuclear receptor and the helper protein. Non-limiting examples of combinations of nuclear receptors and helper proteins are provided in Table 4, which can be used in any of the embodiments of the assay.

Expression of at least one of the nuclear receptor and/or the helper protein is from a nucleic acid which has been introduced into a cell. In some embodiments, the nuclear receptor and the helper protein are expressed from the same nucleic acid. In other embodiments they are expressed from different nucleic acids. In some embodiments two or more nuclear receptors are expressed in the same cell. In some embodiments two or more helper proteins are expressed in the same cell. In a further embodiment one or more helper proteins is naturally expressed or over-expressed by the cell.

In further embodiments, a marker of cell amplification or alternatively a reporter gene is also expressed in the cell. The marker of cell amplification or the reporter gene can be expressed from a separate nucleic acid from the receptor or helper or can be expressed from the same nucleic acid as the nuclear receptor and/or the helper gene.

In one embodiment, the measurable output is used to identify a change in the receptor activity due to the compound being tested. In one embodiment, the measurable output is a morphological change. Thus, for example, the cells can be transfected with the test substance and the control cells without the test substance compared to those which express the test substance. The cells can be analyzed by microscopy and any morphological change can be identified. In one embodiment, the morphological change occurs due to differentiation of the cells.

In a further embodiment, the cells are analyzed with respect to a change in phosphorylation. The change in phosphorylation can be identified using any method known to one of skill in the art, for example, P³²-labelled ATP can be used in a phosphorylation assay and the amount of radioactive label incorporated into the cell can be analyzed. A change in phosphorylation can be a quantitative change, including increased or decreased phosphorylation or alternatively the change can be a qualitative change such as a change in the molecular weight of the proteins being phosphorylated or a change in the location of the proteins being phosphorylated. In one embodiment, antibodies which recognize phosphorylated proteins can be used. These antibodies can be quantitated and/or analyzed in a variety of ways known to one of skill in the art, including but not limited to, western blot, FACS analysis, and In situ techniques. Changes such as localization of the antibodies, amount and/or pattern can be analyzed. Alternatively, the amount of antibody which binds in a cell extract and or the size of the proteins which bind to the antibodies can be analyzed. In other embodiments, the phosphorylation of proteins within the cell in response to the level of a nuclear receptor can be assessed using two dimensional gel electrophoresis.

In a further embodiment, gene expression can be used as the measurable output. In one embodiment, a reporter gene is used. The reporter gene can be expressed from a promoter containing the hormone response elements specific for at least one nuclear hormone receptor in the assay. In some embodiments, the reporter construct can be transfected into a cell along with a nucleic acid encoding at least one receptor and a nucleic acid encoding at least one helper protein (a ‘helper gene’) and the cell can be contacted with one or more test substances. Any or all of the genes which are transfected can be on the same nucleic acid or separate nucleic acids. The measurable output, which in this case is correlated with the level of the transcription of the reporter can then be quantified with and without the test substance.

### Definitions

In the present description and claims, the following terms shall be defined as indicated below.

A “test substance” and/or “candidate compound” is intended to include any drug, compound or molecules with potential biological activity. The test substance can be any substance which can functionally interact with the nuclear hormone receptor in combination with a helper gene.

A “ligand” is intended to include any substance that either inhibits or stimulates the activity of a receptor. An “agonist” is defined as a ligand increasing the functional activity of a receptor (i.e. signal transduction through the receptor). An “antagonist” is defined as a ligand decreasing the functional activity of a receptor either by inhibiting the action of an agonist or by its own activity (inverse agonist). A “selective modulator” is defined as a ligand that modulates the activity of a particular combination of a nuclear receptor and one or more polypeptides from a group of coactivators, corepressor, kinase or signaling molecule.

A “modulator” and/or a ‘helper gene’ of nuclear hormone receptors can be any polypeptide which modulates directly or indirectly the activity of a nuclear receptor, including but not limited to a co-repressor, a kinase, a signaling molecule, a co-activator, a peptide and a receptor.

A “nuclear receptor” is intended to include any molecule present inside a cell either in the cytoplasm and/or in the nucleus which affects cellular physiology and further can be inhibited or stimulated by a ligand. Typically, a nuclear receptor comprises one or two transcriptional activation domains (AF-1 and AF-2) that generates a cellular signal in absence (AF-1) or in response (AF-2) to ligand binding, a ligand-binding domain (LBD) with ligand-binding properties, a DNA-binding domain (DBD) that interacts with specific sequences (cis-acting elements) onto the DNA. In addition, a “nuclear receptor” includes a truncated, modified, mutated receptor, or any molecule comprising partial or all of the sequences of a nuclear receptor.

### Embodiments

In some embodiments of each of the methods described herein, the DNA encoding at least one nuclear receptor can comprise a nucleic acid selected from the group consisting of SEQ ID NOs.: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, and 143 or a nucleic acid homologous thereto In some embodiments, the homologous nucleic acid can have at least 97%, at least 95%, at least 90%, at least 85%, at least 80%, or at least 70% nucleotide sequence identity to a nucleotide sequence selected from the group consisting of SEQ ID NOS.: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, and 143. In some embodiments, the homologous nucleic acid can have at least 97%, at least 95%, at least 90%, at least 85%, at least 80%, or at least 70% nucleotide sequence identity to a nucleic acid comprising at least 10, 15, 20, 25, 30, 35, 40, 50, 75, 100, 150, 200, 300, 400, or 500 consecutive nucleotides of one of SEQ ID NOs.: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, and 143. Identity can be measured using BLASTN version 2.0 with the default parameters or tBLASTX with the default parameters. (Altschul, S. F. et al. Gapped BLAST and PSI-BLAST: A New Generation of Protein Database Search Programs, Nucleic Acid Res. 25: 3389-3402 (1997), the disclosure of which is incorporated herein by reference in its entirety) Alternatively, in some embodiments, the homologous nucleic acid can be a nucleic acid which is in a functional ortholog cluster which contains a nucleic acid selected from the group consisting of SEQ ID NOs.: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, and 143. All other members of that ortholog cluster would be considered homologues. An exemplary library of functional ortholog clusters can be found at The National Center for Biotechnology website at the National Library of Medicine of the National Institutes of Health, which can be accessed on the internet by entering the following quoted text “www.ncbi.nim.nih” in the address bar of a web browser, such as INTERNET EXPLORER™ or NETSCAPE™ followed immediately by “.gov/COG”. Genes can be classified into clusters of orthologous groups or COG by using the COGNITOR program available at the National Center for Biotechnology website above, or by direct BLASTP comparison of the gene of interest to the members of the COGs and analysis of these results as described by Tatusov, R. L., Galperin, M. Y., Natale, D. A. and Koonin, E. V. (2000) The COG database: a tool for genome-scale analysis of protein functions and evolution. Nucleic Acids Research v. 28 n. 1, pp. 33-36.

In some embodiments of each of the methods described herein, the DNA encoding the nuclear receptor comprises nucleotide sequences which encode polypeptides having at least 99%, 95%, at least 90%, at least 85%, at least 80%, at least 70%, at least 60%, at least 50%, at least 40% or at least 25% amino acid identity or similarity to the amino acid sequence of one of SEQ ID NOs: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, and 144 or to fragments comprising at least 5, 10, 15, 20, 25, 30, 35, 40, 50, 75, 100, or 150 consecutive amino acids thereof as determined using the FASTA version 3.0t78 algorithm with the default parameters. Alternatively, protein identity or similarity can be identified using BLASTP with the default parameters, BLASTX with the default parameters, TBLASTN with the default parameters, or tBLASTX with the default parameters. (Altschul, S. F. et al. Gapped BLAST and PSI-BLAST: A New Generation of Protein Database Search Programs, Nucleic Acid Res. 25: 3389-3402 (1997), the disclosure of which is incorporated herein by reference in its entirety).

In some embodiments of each of the methods described herein, the DNA encoding at least one nuclear receptor comprises a DNA which hybridizes under stringent or moderate conditions to a nucleic acid selected from the group consisting of the nucleotide sequences complementary to one of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, and 143. In some embodiments of each of the methods described herein the DNA encoding the nuclear receptor comprises a DNA which hybridizes under stringent or moderate conditions to a fragment comprising at least 10, 15, 20, 25, 30, 35, 40, 50, 75, 100, 150, 200, 300, 400, or 500 consecutive nucleotides of the sequences complementary to a nucleic acid coding for a nuclear hormone receptor including but not limited to one of SEQ ID NOs.: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, and 143. As used herein, “stringent conditions” means hybridization to filter-bound nucleic acid in 6×SSC at about 45° C. followed by one or more washes in 0.1×SSC/0.2% SDS at about 68° C. Other exemplary stringent conditions can refer, e.g., to washing in 6×SSC/0.05% sodium pyrophosphate at 37° C., 48° C., 55° C., and 60° C. as appropriate for the particular probe being used. As used herein, “moderate conditions” means hybridization to filter-bound DNA in 6×sodium chloride/sodium citrate (SSC) at about 45° C. followed by one, preferably 3-5 washes in 0.2×SSC/0.1% SDS at about 42-65° C.

In some embodiments of each of the methods described herein, the DNA encoding the nuclear receptor comprises a DNA which encodes a polypeptide having at least 99%, 95%, at least 90%, at least 85%, at least 80%, at least 70%, at least 60%, at least 50%, at least 40% or at least 25% amino acid identity or similarity to a sequence selected from the group consisting of SEQ ID NOs: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, and 144. In some embodiments of each of the methods described herein, the DNA encoding a nuclear receptor comprises a DNA which encodes a polypeptide having at least 99%, 95%, at least 90%, at least 85%, at least 80%, at least 70%, at least 60%, at least 50%, at least 40% or at least 25% amino acid identity or similarity to a fragment comprising at least 5, 10, 15, 20, 25, 30, 35, 40, 50, 75, 100, or 150 consecutive amino acids of a sequence selected from the group consisting of SEQ ID NOs.: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, and 144. Identity or similarity can be determined using the FASTA version 3.0t78 algorithm with the default parameters. Alternatively, protein identity or similarity can be identified using BLASTP with the default parameters, BLASTX with the default parameters, or TBLASTN with the default parameters. (Altschul, S. F. et al. Gapped BLAST and PSI-BLAST: A New Generation of Protein Database Search Programs, Nucleic Acid Res. 25: 3389-3402 (1997), the disclosure of which is incorporated herein by reference in its entirety).

Alternatively, the nuclear receptor can be a mutant nuclear receptor and can be used in the assay to compare the activity of the mutant to the activity of the wildtype receptor.

In some embodiments of each of the methods described herein where DNA encoding one or more helper proteins in addition to DNA encoding a nuclear receptor are expressed, the DNA encoding one or more helper proteins can comprise a DNA a sequence for a ‘helper gene’ selected from the group consisting of SEQ ID NOs.: 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, and 241 or a DNA which has at least 97%, at least 95%, at least 90%, at least 85%, at least 80%, at least 70% , at least 60%, at least 50%, or at least 40% nucleotide sequence identity to a nucleotide sequence selected from the group consisting of SEQ ID NOs.: 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, and 241. In another embodiment, the DNA encoding one or more helper proteins can comprise a DNA sequence which has at least 97%, at least 95%, at least 90%, at least 85%, at least 80%, at least 70% , at least 60%, at least 50%, or at least 40% nucleotide sequence identity to a nucleic acid comprising at least 10, 15, 20, 25, 30, 35, 40, 50, 75, 100, 150, 200, 300, 400, or 500 consecutive nucleotides of one of SEQ ID NOs.: 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, and 241. Identity can be measured using BLASTN version 2.0 with the default parameters or tBLASTX with the default parameters. (Altschul, S. F. et al. Gapped BLAST and PSI-BLAST: A New Generation of Protein Database Search Programs, Nucleic Acid Res. 25: 3389-3402 (1997), the disclosure of which is incorporated herein by reference in its entirety) Alternatively, the DNA encoding the one or more helper proteins can comprise a nucleic acid which is included in a functional ortholog cluster which contains a nucleic acid selected from the group consisting of SEQ ID NOs.: 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, and 241. All other members of that ortholog cluster can be used in each of the methods described herein. Such a library of functional ortholog clusters can be found at The National Center for Biotechnology website at the National Library of Medicine of the National Institutes of Health, which can be accessed on the internet by entering the following quoted text “www.ncbi.nim.nih” in the address bar of a web browser, such as INTERNET EXPLORER™ or NETSCAPE™ followed immediately by “.gov/COG”. A gene can be classified into a cluster of orthologous groups or COG by using the COGNITOR program available at the same web site, or by direct BLASTP comparison of the gene of interest to the members of the COGs and analysis of these results as described by Tatusov, R. L., Galperin, M. Y., Natale, D. A. and Koonin, E. V. (2000) The COG database: a tool for genome-scale analysis of protein functions and evolution. Nucleic Acids Research v. 28 n. 1, pp. 33-36.

In some embodiments of each of the methods described herein where DNA encoding one or more helper proteins in addition to DNA encoding a nuclear receptor are expressed, the DNA encoding the one or more helper proteins can comprise a DNA which encodes a polypeptide having at least 99%, 95%, at least 90%, at least 85%, at least 80%, at least 70%, at least 60%, at least 50%, at least 40% or at least 25% amino acid identity or similarity to a polypeptide comprising the amino acid sequence of one of SEQ ID NOs: 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, and 242 or to fragments comprising at least 5, 10, 15, 20, 25, 30, 35, 40, 50, 75, 100, or 150 consecutive amino acids thereof as determined using the FASTA version 3.0t78 algorithm with the default parameters. Alternatively, protein identity or similarity can be identified using BLASTP with the default parameters, BLASTX with the default parameters, TBLASTN with the default parameters, or tBLASTX with the default parameters. (Altschul, S. F. et al. Gapped BLAST and PSI-BLAST: A New Generation of Protein Database Search Programs, Nucleic Acid Res. 25: 3389-3402 (1997), the disclosure of which is incorporated herein by reference in its entirety).

In some embodiments of each of the methods described herein where DNA encoding one or more helper proteins in addition to DNA encoding one or more nuclear receptors are expressed, the DNA encoding the one or more helper proteins can comprise a DNA which hybridizes under stringent or moderate conditions to a nucleic acid selected from the group consisting of the nucleotide sequences complementary to one of SEQ ID NOs: 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, and 241. In some embodiments, the DNA encoding the one or more helper proteins can comprise a DNA which hybridizes under stringent or moderate conditions to a fragment comprising at least 10, 15, 20, 25, 30, 35, 40, 50, 75, 100, 150, 200, 300, 400, or 500 consecutive nucleotides of the sequences complementary to one of SEQ ID NOS.: 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, and 241. As used herein, “stringent conditions” means hybridization to filter-bound nucleic acid in 6×SSC at about 45° C. followed by one or more washes in 0.1×SSC/0.2% SDS at about 68° C. Other exemplary stringent conditions can refer, e.g., to washing in 6×SSC/0.05% sodium pyrophosphate at 37° C., 48° C., 55° C., and 60° C. as appropriate for the particular probe being used. As used herein, “moderate conditions” means hybridization to filter-bound DNA in 6×sodium chloride/sodium citrate (SSC) at about 45° C. followed by one, preferably 3-5 washes in 0.2×SSC/0.1% SDS at about 42-65° C.

In some embodiments, the DNA may encode a portion of any of the foregoing nuclear receptors or helper proteins which retains the activity of the nuclear receptor or the helper protein. For example, in some embodiments, the DNA may encode a polypeptide comprising at least 20, at least 30, at least 40, at least 50, at least 75, at least 100, at least 150, at least 200, at least 250, at least 300, at least 350 or more than 350 consecutive amino acids of the nuclear receptor or the helper protein which retains the ability of nuclear receptor or helper protein to perform one or more of its normal physiological functions. Such physiological functions can include but are not limited to transcriptional activation, signaling effects, phosphorylation, interaction with other proteins.

In some embodiments, specific combinations of receptors and helper proteins are used in each of the methods of the present invention. In one embodiment, the combinations include, but are not limited to the combinations selected from the group consisting of: TR beta and DRIP 205 or ERK2; RAR beta and SRC1, DRIP205 or ERK2; PPAR gamma and SRC1 or ERK2; FXR and SRC1, DRIP205 or ERK2; LXR beta and SRC1 or ERK2; VDR and SRC1; PXR and SRC1; RXR alpha and DRIP205 or ERK2; ER beta and SRC1, DRIP205 or ERK2; AR and DRIP205; MR and SRC1 or DRIP205; GR and SRC1; SHP and SRC1 or ERK2; RevERb alpha and ERK2; ROR gamma and DRIP205; HNF4 alpha and SRC1, DRIP205 or ERK2; TR2 alpha and SRC1; TLX and ERK2; COUP-TF beta and SRC1 or DRIP205; EAR2 and SRC1, DRIP205 or ERK2; ERR gamma and ERK2; NOR-1 and SRC1, DRIP205 or ERK2; and GCNF and SRC1 or ERK2.

In a further embodiment, nucleic acid sequences encoding the one or more nuclear receptors and the one or more helper proteins are selected from the group consisting of: SEQ ID NO: 5 and SEQ ID NOs: 161 or 213; SEQ ID NO: 9 and SEQ ID NOs: 145, 147, 161, or 213; SEQ ID NOs: 21, 23, 25, or 27 and SEQ ID NOs: 145, 147, or 213; SEQ ID NO: 49 and SEQ ID NOs: 145, 147, 161, or 213; SEQ ID NO: 45 and SEQ ID NOs: 145 or 213; SEQ ID NO: 51 and SEQ ID NOs: 145 or 147; SEQ ID NOs: 53, 55, or 57 and SEQ ID NOs: 145 or 147; SEQ ID NO: 69 and SEQ ID NOs: 161 or 213; SEQ ID NO: 93 and SEQ ID NOs: 145, 147, 161, or 213; SEQ ID NO: 107 and SEQ ID NO: 161; SEQ ID NO: 103 and SEQ ID NOs: 145, 147, or 161; SEQ ID NO: 101 and SEQ ID NOs: 145 or 147; SEQ ID NO: 143 and SEQ ID NOs: 145, 147 or 161; SEQ ID NO: 29 and SEQ ID NO: 213; SEQ ID NO: 43 and SEQ ID NO: 161; SEQ ID NO: 61 and SEQ ID NOs: 145, 147, 161 or 213; SEQ ID NO: 75 and SEQ ID NOs: 145 or 147; SEQ ID NO: 79 and SEQ ID NO: 161; SEQ ID NO: 87 and SEQ ID NO: 145, 147, or 161; SEQ ID NO: 89 and SEQ ID NOs: 145, 147 or 161; SEQ ID NO: 99 and SEQ ID NO:161; SEQ ID NOs: 123, 125, or 127 and SEQ ID NOs: 145, 147, 161, or 213; and SEQ ID NO: 135 and SEQ ID NOs: 145, 147 or 213.

In a further embodiment, amino acid sequences of said one or more nuclear receptors and said one or more helper proteins are selected from the group consisting of: SEQ ID NO: 6 and SEQ ID NOs: 162 or 214; SEQ ID NO: 10 and SEQ ID NOs: 146, 148, 162, or 214; SEQ ID NOs: 22, 24, 26 or 28 and SEQ ID NOs: 146, 148, or 214; SEQ ID NO: 50 and SEQ ID NOs: 146, 148, 162, or 214; SEQ ID NO: 46 and SEQ ID NOs: 146, 148 or 214; SEQ ID NO: 52 and SEQ ID NOs: 146 or 148; SEQ ID NOs: 54, 56, or 58 and SEQ ID NOs: 146 or 148; SEQ ID NO: 70 and SEQ ID NOs: 162 or 214; SEQ ID NO: 94 and SEQ ID NOs: 146, 148, 162, or 214; SEQ ID NO: 108 and SEQ ID NO: 162; SEQ ID NO: 104 and SEQ ID NOs: 146, 148, or 162; SEQ ID NO: 102 and SEQ ID NOs: 146 or 148; SEQ ID NO: 144 and SEQ ID NOs: 146, 148, or 162; SEQ ID NO: 30 and SEQ ID NO: 214; SEQ ID NO: 44 and SEQ ID NO: 162; SEQ ID NO: 62 and SEQ ID NOs: 146, 148, 162, or 214; SEQ ID NO: 76 and SEQ ID NOs: 146 or 148; SEQ ID NO: 80 and SEQ ID NO: 162; SEQ ID NO: 89 and SEQ ID NO: 146, 148, or 162; SEQ ID NO: 90 and SEQ ID NOs: 146, 148, or 162; SEQ ID NO: 100 and SEQ ID NO:162; SEQ ID NOs: 124, 126, or 128 and SEQ ID NOs: 146, 148, 162, or 214; and SEQ ID NO: 136 and SEQ ID NOs: 146, 148 or 214.

Additional embodiments of the present invention are described in Appendix A which is being filed along with the present application.

### Description

### Nuclear Receptors

The nuclear receptor family includes receptors for classic endocrine hormones, such as estrogens, androgens, glucocorticoids, T3/T4 thyroid hormones, retinoids, and vitamin D₃. As a group, they include a wide variety of nuclear receptors that respond to a plethora of small hydrophobic ligands and control a corresponding assortment of target genes. The nuclear receptor family also contains members that harken back to their primordial ancestors and respond to intermediates in lipid metabolism rather than endocrine hormones per se; examples of the latter include the peroxisome proliferator-activated receptors (PPARs), liver X receptor (LXR), and farnesoid X receptors (FXRs). Finally, there are orphan receptors, such as the chicken ovalbumin upstream regulatory sequence transcription factors (COUP-TFs), for which no ligands have been identified.

Exempting the orphan receptors, the generic nuclear receptor operates as a single-step signal transducer, transmitting an input (the binding of a small chemical ligand) into an output (such as a change in the transcription rate of specific target genes). In many pathways, but without being limited to a specific pathway, to do so, the nuclear receptor (a) recognizes the specific DNA sequences, denoted hormone response elements (HREs), in or near the target gene, (b) binds to the hormone or lipid ligand, and ultimately (c) mediates the molecular events that alter the rate of transcription of the target promoter.

Briefly, most nuclear receptors bind to DNA either as homodimers or as heterodimers with other members of the nuclear receptor family (especially with the RXR members), a few can also recognize DNA as receptor monomers, or as oligomers. A zinc-finger motif in each receptor monomer recognizes a six to eight nucleotide sequence on the DNA, denoted a half site. To recruit a receptor dimer, a functional HRE contains two half sites arranged in a specific orientation and spacing. For instance, thyroid hormone receptors (T3Rs) preferentially bind to two AGGTCA half sites oriented as direct repeats with a four-base spacer (DR-4s); retinoic acid receptors (RARs) bind to the same AGGTCA half sites, but oriented as a DR-5; estrogen receptors bind to AGGTCA half sites oriented as an inverted repeat with a three-base spacer (INV-3); and androgen receptors (ARs) recognize an INV-3 orientation containing AGAACA half sites. This precis is necessarily a simplification: HREs in nature often contain half sites that diverge in sequence and topology from these prototypic elements. Nuclear receptors also interact with nonreceptor transcription factors, such as c-Jun and c-Fos, either to tether the receptor indirectly to the DNA or to form complexes in which both the receptor and nonreceptor contribute specific DNA contacts. These interactions can result in complex, combinatorial modes of transcriptional regulation.

Much elegant work has also been devoted to understanding how nuclear receptors recognize their hormone ligands. The operative entity in this regard is a C-terminal hormone-binding domain (HDB) composed of 12 α-helical domains twisted into a triple-layered sandwich. Hormone ligand is virtually engulfed by this polypeptide sandwich, with the hormone serving as a hydrophobic core on which the receptor completes its own folding. Due to this close approximation between ligand and receptor, different hormones can invoke different conformations in the receptor. These ligand-driven receptor conformations produce distinct biological consequences. For example, ligand agonists produce receptor conformations that favor transcriptional activation, whereas ligand antagonists produce receptor conformations that favor transcriptional repression.

Nuclear receptors operate by recruiting an array of auxiliary polypeptides, denoted corepressors and coactivators, and it is these auxiliary proteins that mediate the molecular events that result in transcriptional repression or activation. The molecular basis of this transcriptional drama is described in greater detail below.

Nuclear receptors possess subdomains that are used for transcriptional regulation, yet can be distinguished from sequences used for DNA binding or hormone recognition. These transcriptional regulatory domains have several aliases (activation domains, activation function domains, tau domains, repression domains, silencing domains) but a common mode of operation; they represent docking surfaces on the receptor through which corepressors and coactivators are recruited. Almost all nuclear receptors possess a hormone-dependent activation domain in the receptor HBD; this activation function (AF)-2 receptor domain forms a docking surface for coactivators and is assembled in three-dimensional space from portions of HDB helices 3/5/6 and 12. Intriguingly, this same surface overlaps an important corepressor binding site, and a yin yang mechanism operates by which hormone-induced changes in HBD helix 12 alternatively favor recruitment for one or the other class of coregulator. Many, but not all, nuclear receptors possess additional activation domains within their N-terminal domains (denoted AF-1 sequences) that bind coactivators, as well as less-characterized corepressor and coactivator interaction surfaces within their DNA-binding domains.

By exploiting these various docking surfaces as bait in two-hybrid or in coprecipitation experiments, researchers have compiled an increasingly thick dossier of coactivators and corepressors. The coactivators thus identified can be broadly categorized into four groups: (a) histone covalent modifiers, such as the P160 family, CARM, and CBP/p300, that possess (or recruit) enzymatic activities able to modify the chromatin template, including acetylases and methylases; (b) ATP-dependent chromatin-remodeling complexes, such as the Swi/Snf family, that alter the higher-order structure and position of nucleosomes; (c) components of the mediator complex, such as TRAP/DRIP, that interact with the general transcriptional machinery to assist in assembly of the preinitiation complex; and (d) coactivators with unknown functions.

The first corepressors identified for nuclear receptors were SMRT (also known as the T3R-associated cofactor, TRAC) and its close paralog, N—CoR (also known as the receptor interacting protein 13, RIP 13). SMRT and N—CoR are encoded by two distinct loci but share a common molecular architecture and approximately 45% amino acid identity, additional forms of SMRT and N—CoR are generated by alternative mRNA splicing, and including SMRTτ.

Both SMRT and N—CoR can be conceptually divided into a N-terminal portion having three to four distinct transcriptional repression (or silencing) domains (RDs), and a C-terminal portion composed of two or three nuclear receptor interaction domains (NDs). The RDs are docking surfaces that recruit additional components of the corepressor complex, including histone deacetylases (HDACs), transducin-like protein 1 (TBL-1), G protein pathway suppressor 2 (GPS2), and (possibly) mSin3 and its cohorts.

Receptor homodimers and heterodimers can display different N—CoR— and SMRT-binding properties. For example, T3Rs homodimers, but not T3R/RXR heterodimers, efficiently recruit SMRT and N—CoR when bound to DNA response elements and can be important mediators of T3R repression.

A third subgroup of nuclear receptors display low or no corepressor binding in the absence of hormones, but gain an increased ability to bind corepressors in the presence of hormone antagonists: these include estrogen receptors (ERs), glucocorticoid receptors (GRs), progesterone receptors (PRs), and androgen receptors (ARs).

Many nuclear receptors are expressed from multiple genetic loci, or by alternative mRNA splicing, to generate multiple receptor isotypes (or isoforms) that play distinct roles in development and physiology. These receptor isotypes can display different corepressor recruitment properties. For example, RARs are encoded by three distinct genes: α, β, and γ. Although RARE represses target gene expression in the absence of hormone, RARβ and γ do not repress, rather they activate transcription in both the absence and presence of hormone agonist. These differences in transcriptional regulation reflect the corepressor binding properties of these isoforms: RARE binds corepressor strongly in vitro and in vivo, whereas RARβ and γ do not.

Transfection of cells in the present invention can be performed according to any one of the numerous methods known in the art. In general, DNA sequences encoding one or more nuclear receptor and DNA sequences encoding coactivators, corepressors, kinases and other signaling molecules can be inserted in suitable cloning vectors that can conveniently be subjected to recombinant DNA procedures. Expression vectors carry promoter sequences that allow the expression of the nuclear receptor, coactivator, corepressor, kinase or other signaling molecules. The promoter can be any DNA sequence that shows transcriptional activity in the host cell of choice and can be derived from gene encoding proteins either homologous or heterologous to the host cell. The vector can also comprise elements such as polyadenylation signals, transcriptional enhancer sequences, translational enhancer sequences, origin of replication and integration sequences. The procedures used to insert the DNA sequences into suitable vectors are well known to those skilled in the art.

In further embodiments, cells can be transfected with at least one other nuclear receptor that is known to, suspected to, or will be tested to determine whether it will heterodimerize with the first nuclear receptor. In this way one can identify ligands, agonists, antagonists, inverse agonists or selective modulators that specifically interact with the heterodimer.

Examples of suitable promoters for directing the transcription of the DNA encoding the nuclear receptor and/or ‘helper genes’ such as genes encoding, coactivators, corepressors, kinases or other signaling molecules in mammalian cells, include but are not limited to: the SV40 promoter (Subramani et al., Mol. Cell Biol. 1 (1981), 854-864), the MT-1 (metallothionein gene) promoter Palmiter et al., Science 222 (1983), 809-814) or adeno-virus 2 major late promoter.

The DNA sequence encoding the nuclear receptor, helper proteins such as, coactivators, corepressors, kinases or other signaling molecules can also be operably connected to a suitable terminator, such as the human growth hormone terminator (Palmiter et al. op. cit.). The vector can further comprise elements such as polyadenylation signals (e.g. from SV40 or the adenovirus 5 Elb region), transcriptional enhancer sequences (e.g., the SV40 enhancer) and translation enhancer sequences (e.g., the ones encoding adenovirus VA RNAs).

The vector can further comprise a DNA sequence enabling the vector to replicate in the host cell in question. An example of such a sequence (when the host cell is a mammalian cell) is the SV40 origin of replication.

The procedures used to ligate the DNA sequences coding for the receptor, the promoter and the terminator, respectively, and to insert them into suitable vectors containing the information necessary for replication, are well known to persons skilled in the art (cf., for instance, Sambrook et al., Molecular Cloning: A Laboratory Manual, Cold Spring Harbor Laboratory, Cold Spring Harbor, N.Y., 1989).

Cells that can be used in the present method include any cells capable of mediating signal transduction via the nuclear receptor in the presence of one or more ‘helper genes’ encoding helper proteins such as coactivators, corepressors, kinases, or signaling molecules. Such cells are typically mammalian cells but eukaryotic (such as insect cells) or prokaryotic cells are also suitable. Examples of useful mammalian cells that can be used include, but are not limited to: the preferred mouse fibroblastic cell line NIH-3T3 (ATCC CRL 1658), RAT 1 cells, HEK 293 cells, CHO cells and COS cells.

Methods of transfecting mammalian cells and expressing DNA sequences introduced in the cells are described in e.g., Kaufman and sharp, J. Mol. Biol. 159 (1982), 601-621; Southern and Berg, J. Mol. Appl. Genet. 1 (1982), 327-341; Loyter et al., Proc. Natl. Acad. Sci USA 79 (1982), 422-426; Wigler et al., Cell 14 (1978), 725; Corsaro and Pearson, Somatic Cell Genetics 7 (1981), 603, Graham and ver der Eb, Virology 52 (1973), 456; Neumann et al., EMBO J. 1 (1982), 841-845; and Wigler et al., Cell 11, 1977, pp. 223-232.

Any nuclear hormone receptors known to one of skill in the art can be utilized in the present invention, including but not limited to: those in Tables 1 and 3, those described above under the heading “nuclear hormone receptors”, and those that are newly identified as nuclear hormone receptors. Although most of the nuclear hormone receptors specifically referred to herein are human, it will be appreciated that one could perform the assay with homologs of any of these receptors, such as mammalian, insect and other homologs of these receptors, some of which have already been identified. Homologs include anything with from about 30%-100% amino acid identity, including but not limited to 35%, 40%, 45%, 50%, 55%, 60%, 65%, 70%, 75%, 80%, 85%, 90%, 95%, and 99% of any human nuclear hormone receptor. Amino acid identity can be determined using any of the conventional software, including BLAST. Alternatively, the homology can be to the SEQ ID NOs disclosed herein, including but not limited to SEQ ID NOs: 1-48. The homologs can be identified using any methods known to one of skill in the art.

In some embodiments, nuclear hormone receptors for which activity can be modulated in a ligand dependent manner can be assayed in the present invention by co-expressing one or more signaling molecules in the cells expressing the nuclear hormone receptor. Signaling molecules can be any molecules that directly or indirectly modulate the activity of the nuclear receptor or receptors, including but not limited to: coactivators, corepressors, kinases, signaling molecules.

TABLE 1
List of human nuclear hormone receptors

| | | Unifying | GenBank # - SEQ ID | | | |
|---|---|---|---|---|---|---|
| | Gene | name | Nos) | Variant 2 | Variant 3 | Variant 4 |
| Group I | | | | | | |
| 1A | NR1A1 | TR alpha | NM 199334 - 1, 2 | NM 003250 - 3, 4 | | |
| | NR1A2 | TR beta | NM 000461 - 5, 6 | | | |
| 1B | NR1B1 | RAR alpha | NM 000964 - 7, 8 | | | |
| | NR1B2 | RAR beta | NM 000965 - 9, 10 | NM 016152 - 11, | | |
| | | | | 12 | | |
| | NR1B3 | RAR gamma | NM 000966 - 13, 14 | | | |
| 1C | NR1C1 | PPAR alpha | NM 005036 - 15, 16 | | | |
| | NR1C2 | PPAR | NM 006238 - 17, 18 | NM 177435 - 19, | | |
| | | beta/delta | | 20 | | |
| | NR1C3 | PPAR gamma | NM 005037 - 21, 22 | NM 015869 - 23, | NM 138711 - | NM 138712 - |
| | | | | 24 | 25, 26 | 27, 28 |
| 1D | NR1D1 | RevERB alpha | NM 021724 - 29, 30 | | | |
| | NR1D2 | RevERB beta | NM 005126 - 31, 32 | | | |
| 1F | NR1F1 | ROR alpha | NM 134261 - 33, 34 | NM 134262 - 35, | NM 134260 - | NM 002493 - |
| | | | | 36 | 37, 38 | 39, 40 |
| | NR1F2 | ROR beta | NM 006914 - 41, 42 | | | |
| | NR1F3 | ROR gamma | NM 005060 - 43, 44 | | | |
| 1H | NR1H2 | LXR beta | NM 007121 - 45, 46 | | | |
| | NR1H3 | LXR alpha | NM 005693 - 47, 48 | | | |
| | NR1H4 | FXR | NM 005123 - 49, 50 | | | |
| 1I | NR1I1 | VDR | NM 000376 - 51, 52 | | | |
| | NR1I2 | PXR | NM 003889 - 53, 54 | NM 022002 - 55, | NM 033013 - | |
| | | | | 56 | 57, 58 | |
| | NR1I3 | CAR alpha | NM 005122 - 59, 60 | | | |
| Group II | | | | | | |
| 2A | NR2A1 | HNF4 alpha | NM 178849 - 61, 62 | NM 000457 - 63, | NM 178850 - | |
| | | | | 64 | 65, 66 | |
| | NR2A2 | HNF4 gamma | NM 004133 - 67, 68 | | | |
| 2B | NR2B1 | RXR alpha | NM 002957 - 69, 70 | | | |
| | NR2B2 | RXR beta | NM 021976 - 71, 72 | | | |
| | NR2B3 | RXR gamma | NM 006917 - 73, 74 | | | |
| 2C | NR2C1 | TR2 alpha | NM 003297 - 75, 76 | | | |
| | NR2C2 | TR2 beta | NM 003298 - 77, 78 | | | |
| 2E | NR2E1 | TLX | NM 003269 - 79, 80 | | | |
| | NR2E3 | PNR | NM 016346 - 81, 82 | NM 014249 - 83, | | |
| | | | | 84 | | |
| 2F | NR2F1 | COUP-TF | NM 005654 - 85, 86 | | | |
| | | alpha | | | | |
| | NR2F2 | COUP-TF beta | NM 021005 - 87, 88 | | | |
| | NR2F6 | EAR2 | XM 373407 - 89, 90 | | | |
| Group III | | | | | | |
| 3A | NR3A1 | ER alpha | NM 000125 - 91, 92 | | | |
| | NR3A2 | ER beta | NM 001437 - 93, 94 | | | |
| 3B | NR3B1 | ERR alpha | NM 004451 - 95, 96 | | | |
| | NR3B2 | ERR beta | NM 004452 - 97, 98 | | | |
| | NR3B3 | ERR gamma | NM 001438 - 99, 100 | | | |
| 3C | NR3C1 | GR alpha | NM 000176 - 101, 102 | | | |
| | NR3C2 | MR | NM 000901 - 103, 104 | | | |
| | NR3C3 | PR | NM 000926 - 105, 106 | | | |
| | NR3C4 | AR | NM 000044 - 107, 108 | | | |
| Group IV | | | | | | |
| 4A | NR4A1 | Nurr77 | NM 002135 - 109, 110 | NM 173157 - | NM 173158 - | |
| | | | | 111, 112 | 113, 114 | |
| | NR4A2 | Nurr1 | NM 006186 - 115, 116 | NM 173171 - | NM 173172 - | NM 173173 - |
| | | | | 117, 118 | 119, 120 | 121, 122 |
| | NR4A3 | Nor1 | NM 006981 - 123, 124 | NM 173198 - | NM 173199 - | NM 173200 - |
| | | | | 125, 126 | 127, 128 | 129, 130 |
| Group V | | | | | | |
| 5A | NR5A1 | SF-1 | NM 004959 - 131, 132 | | | |
| | NR5A2 | LRH-1 | NM 003822 - 133, 134 | | | |
| Group VI | | | | | | |
| 6A | NR6A1 | GCNF | NM 033334 - 135, 136 | NM 001489 - | NM 033335 - | |
| | | | | 137, 138 | 139, 140 | |
| Group VII | | | | | | |
| 0B | NR0B1 | DAX1 | NM 000475 - 141, 142 | | | |
| | NR0B2 | SHP | NM 021969 - 143, 144 | | | |

The sequences of all the GenBank Accession Numbers in Table 1 are incorporated herein by reference. The sequences are designated with the accession number followed by the SEQ ID NO: for the nucleotide sequence followed by the protein sequence. For example NM199334—1, 2 means that the nucleotide sequence for NR1A1 (TR alpha) is SEQ ID NO:1 and the protein sequence is SEQ ID NO:2.

In embodiments employing known ligands, the ligands can be any ligand that binds to the nuclear hormone receptor and is known to one of skill in the art. Examples of known ligands include but are not limited to those in Table 3 that also identify the nuclear hormone receptor they are associated with.

In some embodiments, one or more ‘helper genes’ encoding helper proteins including but not limited to: coactivators, corepressors, kinases and/or signaling molecules, are expressed in the cells expressing the nuclear receptor. Alternatively, in some embodiments, polypeptides to be tested for activity as a coactivator, kinase or signaling molecule are expressed in the cells expressing the receptor. In embodiments employing the known ‘helper genes’ (encoding helper proteins such as coactivators, corepressors, kinases or signaling molecules), any such molecules can also be used that are known to one of skill in the art, including but not limited to those identified in Table 2. These coactivators, corepressors or kinases acting as helper proteins can be provided in the cells to be assayed. By providing these molecules one can identify ligands selective for a particular combination of the nuclear receptor and one or more helper genes proteins such as coactivators, corepressors, kinases or signaling molecules. Thus, signaling molecules can be expressed in the cell in addition to one or more receptors and the cells can be contacted with a ligand. Alternatively, the assay can be carried out without contacting the cells with a ligand in embodiments in which the receptor is constitutively active.

TABLE 2
List of modulators of nuclear hormone receptor activity

| Name | Alternate Names | GenBank # | Variant 1 | Variant 2 | Variant 3 | Variant 4 |
|---|---|---|---|---|---|---|
| Co-Activators | | | | | | |
| SRC family | | | | | | |
| SRC-1 | NCoA-1 | NM 003743 - | NM 147223 - | NM 147233 - | | |
| | | 145, 146 | 147, 148 | 149, 150 | | |
| SRC-2 | TIF2, GRIP1, NCoA2 | NM 006540 - | | | | |
| | | 151, 152 | | | | |
| SRC-3 | RAC3, AIB1, ACTR | NM 181659 - | NM 006534 - | | | |
| | | 153, 154 | 155, 156 | | | |
| TRAP/DRIP family | | | | | | |
| DRIP250 | KIAA 0593 | AB 011165 - | | | | |
| | | 157, 158 | | | | |
| DRIP240 | KIAA 0192 | D 83783 - | | | | |
| | | 159, 160 | | | | |
| DRIP205 | TRAP220 | AF 055994 - | | | | |
| | | 161, 162 | | | | |
| DRIP150 | RGR-1 | AF 304448 - | | | | |
| | | 163, 164 | | | | |
| DRIP130 | | AF 105332 - | | | | |
| | | 165, 166 | | | | |
| DRIP100 | TRAP100 | NM 014815 - | | | | |
| | | 167, 168 | | | | |
| DRIP92 | | AF 106934 | | | | |
| | | 169, 170 | | | | |
| DRIP80 | | AF 105421 - | | | | |
| | | 171, 172 | | | | |
| DRIP36 | HSPC126 | AF 161475 - | | | | |
| | | 173, 174 | | | | |
| Misc. | | | | | | |
| CBP | p300 | NM 001429 | | | | |
| | | 175, 176 | | | | |
| PCAF | CAF | NM 003884 - | | | | |
| | | 177, 178 | | | | |
| CARM1 | PRMT4 | NM 199141 - | | | | |
| | | 179, 180 | | | | |
| PGC-1 alpha | PPARGC1, PGC1 | NM 013261 - | | | | |
| | | 181, 182 | | | | |
| PGC-1 beta | PERC, PGC1B | NM 133263 - | | | | |
| | | 183, 184 | | | | |
| HDAC9 | HDAC, HDRP | NM 058176 - | NM 058177 - | NM 014707 - | NM 178423 - | NM 178425 - |
| | | 185, 186 | 187, 188 | 189, 190 | 191, 192 | 193, 194 |
| Co-Repressors | | | | | | |
| NCOR-1 | NCOR | NM 006311 - | AF 303586 - | AF303585 - | AF303584 - | |
| | | 195, 196 | 197, 198 | 199, 200 | 201, 202 | |
| NCOR-2 | SMRT | NM 006312 - | | | | |
| | | 203, 204 | | | | |
| Kinases | | | | | | |
| CDK2 | p33 | NM 001798 - | NM 052827 - | | | |
| | | 205, 206 | 207, 208 | | | |
| CDK7 | | NM 001799 - | | | | |
| | | 209, 210 | | | | |
| ERK1 | MAPK3 | NM 002746 - | | | | |
| | | 211, 212 | | | | |
| ERK2 | MAPK2 | NM 030662 - | | | | |
| | | 213, 214 | | | | |
| GSK-3 | | NM 002093 - | | | | |
| | | 215, 216 | | | | |
| JNK1 | MAPK8 | NM 139049 - | NM 002750 - | NM 139046 - | NM 139047 - | |
| | | 217, 218 | 219, 220 | 221, 222 | 223, 224 | |
| JNK2 | MAPK9 | NM 002752 - | NM 139068 - | NM 139069 - | NM 139070 - | |
| | | 225, 226 | 227, 228 | 229, 230 | 231, 232 | |
| Protein Kinase A | | NM 002730 - | NM 002731 - | NM 002732 - | NM 002733 - | NM 002734 - |
| | | 233, 234 | 235, 236 | 237, 238 | 239, 240 | 241, 242 |

The sequences of all the GenBank Accession Numbers in Table 2 are incorporated herein by reference.

Screening Assays

The screening assay used in the present method can include any functional assay that would reflect one or more nuclear receptor activities in, for instance, mammalian or non-mammalian cells, proteins, cytosolic and/or nuclear extracts, membrane extracts, each of which containing the appropriate nuclear receptor(s), and are capable of sensing the ability of compound(s) to activate or inactivate the receptor(s).

In preferred embodiments, Receptor Selection and Amplification Technology R-SAT (U.S. Pat. Nos. 5,707,798; 5,912,132; and 5,955,281, the disclosures of which are incorporated herein by reference in their entireties), is used as a screening assay.

Other assays involve the initial steps of transfecting an expressible nuclear hormone receptor gene, transfecting at least one “helper gene” and identifying a measurable output in the presence of at least one test substance. In one embodiment, a receptor is selected from Table 1 and at least one helper gene is selected from Table 2. Then at least one test substance can be added and a measurable output analyzed.

For hybridization purposes, DNA can be isolated from the cells and digested with a suitable restriction endonuclease. After digestion, the resulting DNA fragments can be subjected to electrophoresis on an agarose gel. DNA from the gel can then be blotted onto a nitrocellulose filter and hybridized with a radiolabeled oligonucleotide probe. The probe can conveniently contain a DNA fragment of the receptor gene (substantially according to the method of E. M. Southern, J. Mol. Biol. 98, 1975, pp. 503).

For amplification purposes, total mRNA isolated form the cells can be reverse transcribed to prepare a cDNA library. cDNA encoding the receptor can then be amplified by polymerase chain reaction (PCR) using oligonucleotide primers corresponding to segments of the gene coding for receptor in question and detected by size on an agarose gel. Amplified receptor cDNA can also be detected by hybridization to a radiolabelled oligonucleotide probe comprising a DNA sequence corresponding to at least part of the gene encoding the receptor. This method is described by, e.g., Sambrook et al., supra.

Mutant receptors can be used in any of the methods described herein. Such mutant receptors can be used for a variety of reasons, including by not limited to identify corepressors, coactivators, kinases, signaling molecules, agonists and/or antagonists that specifically interact with one form of the receptor such as a homodimeric form, a heterodimeric form, a hormone bound form, an alternatively spliced form, an activated form or a repressed form. Thus, any known site can be mutated in such a way that the other receptor, hormone, coactivator, and/or corepressor can no longer bind. Many such receptors have been identified and produced and are thus known in the art. Alternatively, the methods to produce such a mutant receptor by cloning the receptor, performing mutagenesis on the cloned receptor, and screening for constitutively activated receptors or ligand-independent receptor can be found generally in such references as Maniatis “Molecular Cloning, A Laboratory Manual” and Ausubel et al. “Short Protocols in Molecular Biology”, 1989 Greene Publishing Associates and Wiley-Interscience (See for example, pages 233-250 for mutagenesis methods). Alternatively, the method described herein can be used to screen and identify ligand-independent receptors by comparing the activity of the transfected receptor in the presence and absence of ligand. The presence of the ligand will not affect the activity of the receptor in the assay if the receptor is ligand-independent. In addition, ligand independent receptors can be used in the present methods to identify compounds that inhibit their activity, such as antagonists or inverse agonists.

### EXAMPLES

The invention described and claimed herein is not to be limited in scope by the specific embodiments herein disclosed, since these embodiments are intended as illustration of several aspects of the invention. Any equivalent embodiments are intended within the scope of this invention. Indeed, various modifications of the invention in addition to these shown and described herein will become apparent to those skilled in the art from the foregoing description. Such modifications are also intended to fall within the scope of the appended claims.

The present invention is further disclosed in the following Examples, that are not in any way intended to limit the scope of the invention as claimed.

### Example 1

### A General Protocol for Assaying a Single Nuclear Receptor

The functional cell-based assay Receptor and Selection Amplification Technology (R-SAT™) (U.S. Pat. No. 5,707,798) was modified to develop an assay that allows the investigation of the pharmacological phenotype of one nuclear receptor.

Cultures of NIH-3T3 cells (available from the American Type Culture Collection, as ATCC CRL 1658) were prepared to 50-60% confluency. On day one, cells were trypsinized, centrifuged and plated at 8,000 cells/well in a 96-well plate in 100 μl/well of Dubelcco's Modified Eagle's Medium (DMEM), 10% calf serum. On day two, cells were transfected using the transfection reagent Superfect® (Qiagen, Inc.) as recommended by the manufacturer. Various doses of the nuclear receptor plasmid DNA were transfected. DNA mixtures included 0.1 to 10 ng/well of receptors DNA, 20 to 30 ng/well of β-galactosidase plasmid DNA (pSV β-galactosidase, Promega) and 15 μl of Superfect®. On day three, the media was replaced by DMEM with 2% Cyto-SF3 (Kemp Biotechnologies, Inc.) containing variable amounts of the compounds being tested. Cells were grown at 37° C. in a humidified environment supplemented with 5% CO₂ for five days prior to assessing β-galactosidase activity by replacing the media with the β-galactosidase substrate o-nitrophenyl-β-D-galacto-pyrannoside as described in U.S. Pat. No. 5,707,798. All data were obtained by measuring the change in absorbance at 420 nm using an automated plate reader. EC50 values were calculated using the equation r=A+B(x/(c+c)), where A=minimum response, B=maximum response minus maximum response, c=EC50, r=response, and x=concentration of ligand. Curves were generated using XLFit software (Microsoft).

In the experiments illustrated in Table 3, several nuclear receptors with known ligands were tested using the single receptor format method described above. The data in Table 3 demonstrate that the present methods are useful in obtaining pharmacologically relevant responses to all nuclear hormone receptors.

### Example 2

### Multiple Receptor Format

To test the amenability of the methods described in Example 1 to a multiple receptor format, NIH-3T3 cells were co-transfected with plasmids encoding the glucocorticoid receptor (GR), the Estrogen Receptor beta (ERB), and β-galactosidase cDNA as described in Example 1. The transfected cells were contacted with known ligands for each receptor and activity was measured as described in Example 1. Positive β-galactosidase responses were indicative of effective ligand/receptor interactions. As shown, selective pharmacological responses to the two ligands were seen, confirming that the methods disclosed herein can be used in a multiple receptor format.

### Example 3

### Importance of Co-Activators in the Nuclear Response

The following example demonstrates the importance of co-activators in the pharmacological response of the rifampicin (PXR) receptor (GenBank AF61056). The PXR receptor heterodimerizes with the retinoic acid nuclear receptor RXR subtype (GenBank U38480). The coactivators GRIP1 (Glucocorticoid Receptor Interacting Protein 1) (GenBank U39060) and SRC-1 (Steroid Receptor Coactivator 1) (GenBank U90661) were used in this assay. In summary, plasmid DNA encoding the coactivator(s) were transfected along with the aforementioned transfection mixture (containing the PXR, RXR, and β-Gal plasmid DNAs). The co-transfected cells were contacted with Rifampicin, and β-Galactosidase activity was measured as described in Example 1.

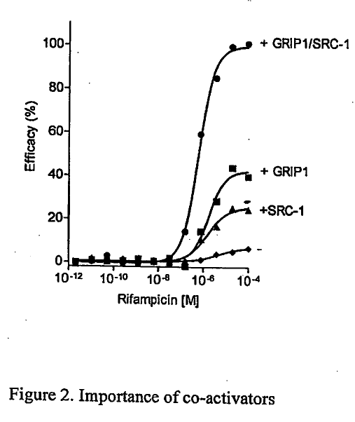

The results are presented in FIG. 2, which represents a typical pharmacological profile of an agonist response of the PXR receptor as determined by R-SAT. The conclusions that can be drawn from this study are:
(a) co-transfection of one co-activator (either SRC-1 or GRIP1) results in the partial activation of the pharmacological PXR response, but significantly stronger than the marginal response observed with PXR and RXR alone,
(b) co-transfection of both co-activators (SRC-1 and GRIP1) results in a synergistic effect that leads to the complete PXR pharmacological response,
(c) co-transfection of multiple co-activators improve the PXR agonist response without displaying any detrimental effects.

The experiments above indicate that co-activators are highly useful in triggering the cellular amplification of cells transfected with the nuclear receptors PXR and RXR, particularly to enough of an extent that they can be easily assayed. Indeed, PXR and RXR expressed alone defined a weak (5-10% efficacy) non-potent pharmacological response. In the presence of co-activators, the PXR/RXR pharmacological response was strong (100% efficacy) and pharmacologically relevant. Moreover, these studies reflect the fact that the amplification assay is highly useful for identifying the signaling requirements (such as co-activators, for example) of nuclear receptors.

### Example 4

### Stimulation of a Number of Nuclear Receptors

Table 3 lists several nuclear receptors belonging to several functional categories based on their ligand-binding properties. Cells transfected with these nuclear reporters were successfully assayed using the general protocol for assaying a single nuclear receptor method described in Example 1, using the indicated ligands. The results are shown in Table 3. These data indicate that all nuclear receptors can be assayed using the methods described herein or any variations of the assay that would be apparent to one of skill in the art.

TABLE 3
Nuclear hormone receptors assayed using R-SAT ™

| Nuclear | Trivial | | EC50 |
|---|---|---|---|
| Receptor | Name | Ligand | nM |
| NR1A2 | TRβ | T3 hormone | 1-2 |
| NR1B1 | RARα | AM-580 | 40-100 |
| NR1B2 | RARβ | AM-580 | 30-100 |
| NR1B2 | RARβ2 | AM-580 | 10-50 |
| NR1B3 | RARγ | AM-580 | 10-50 |
| NR1C2 | PPARδ | carbaprostacyclin | 200-500 |
| NR1H2 | LXRβ | 22(R)OH-Cholesterol | 3,000-5,000 |
| NR1H3 | LXRα | 22(R)OH-Cholesterol | 3,000-5,000 |
| NR1H4 | FXR | CDCA | 1,000-3,000 |
| NR1I1 | VDR | vitamin D3 | 0.05-0.2 |
| NR1I2 | PXR | rifampicin | 500-1,000 |
| NR2B1 | RXRα | retinoic acid | 20-70 |
| NR2B2 | RXRβ | retinoic acid | 20-70 |
| NR2B3 | RXRγ | retinoic acid | 30-100 |
| NR3A1 | Erα | 17 beta estradiol | 0.005-0.030 |
| NR3A2 | Erβ | 17 beta estradiol | 0.005-0.030 |
| NR3C1 | GRα | dexamethasone | 0.1-0.5 |
| NR3C4 | AR | DHT | 0.2-0.5 |

### Example 5

### Detection of Constitutive Activity of Nuclear Receptors

Constitutive activity is defined by the activity that a receptor displays in the absence of binding to an agonist. The following example demonstrates that the amplification assay and methods described herein are useful for determining the constitutive activity of various nuclear receptors. As demonstrated in Example 6, such information is particularly useful in determining experimental parameters to assess, for example, inverse agonist activity of known or unknown compounds on the receptors.

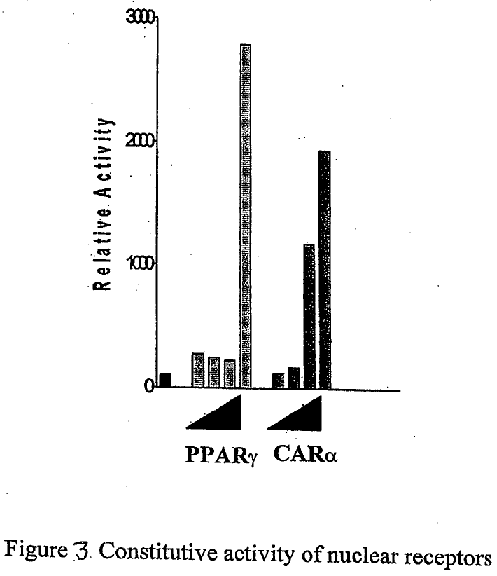

Cells were transfected with plasmid DNAs encoding the RXR retinoic acid receptor and β-Gal reporter, as well as a range of concentrations of plasmid DNA encoding the PPARγ or CARα nuclear receptors as well as as described in Example 1. PPARγ is the peroxisome proliferator activated receptor. CARα is the constitutive androstane receptor (CAR) alpha. Both receptors form heterodimers with the RXR receptor. Transfections were carried out with 300 pg, 1.5 ng, 6.0 ng, or 30 ng of PPARγ or CARα DNA. The β-Galactosidase activity of the transfected cells was measured and compared to cells that did not express a recombinant nuclear receptor, as described in Example 1. FIG. 3 shows the results of the experiments, as expressed in Miller Units.

The conclusions that can be drawn from this study are:
1—The R-SAT™ technology is amenable to measuring levels of constitutive activity displayed by nuclear receptors,
2—Each nuclear receptor expresses different degrees of constitutive activity that are dependent in part upon the quantity of receptor transfected into the cells,
3—The extent of the constitutive activity displayed by a nuclear receptor constitutes a dynamic range that allows for the response to an inverse agonist.

### Example 6

### Detection of Inverse Agonism of Nuclear Receptors

The following example demonstrates that the methods described herein are useful in identifying and detecting inverse agonists of nuclear receptors.

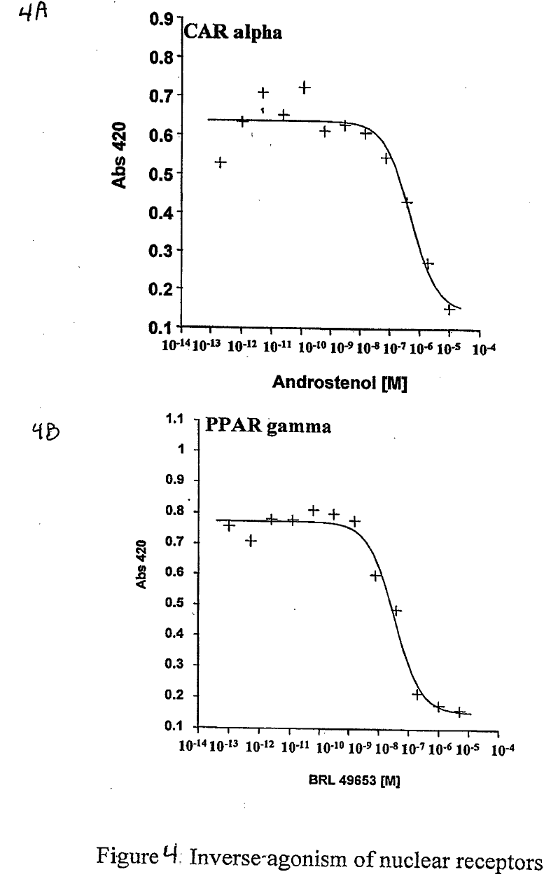

As demonstrated in Example 5, PPARγ and CARα exhibit constitutive activity. Cells were transfected with plasmid DNAs encoding the PPARγ and RXR receptors (known to form heterodimers) or plasmid DNAs encoding CARα and RXR nuclear receptors (known to form heterodimers), as well as the β-Gal reporter DNA as described in Example 1. The cells were contacted with the indicated amounts of known inverse agonists BRL 49653 or Androstenol, respectively, as described in Example 1, and β-Galactosidase activity was measured. The data are presented in FIGS. 4A and 4B, which shows that both compounds had inverse agonist activity. These data demonstrate that the R-SAT™ technology is amenable to detect compounds with inverse agonist activity at nuclear receptor(s).

### Example 7

### Enablement of Receptor Activity Through Different Helper Strategies

The following example illustrates that different helper strategies are needed to enable or improve the activity of nuclear hormone receptors.

Cells were transfected with nuclear receptors with known ligands (Table 4A) or orphan nuclear receptors (Table 4B) and the indicated helper genes (SRC1, GRIP, or Erk2). SRC1 and GRIP are two different types of co-activators, and Erk2 is a kinase. Transfected cells were assayed using R-SAT™ as described in Example 1. Cell samples exhibiting activity of under 500 absorbance units (AU) were designated “−.” Samples exhibiting activity between 100 and 500 units were assigned “+,” samples exhibiting activity between 500 and 1,000 AU were assigned “++,” and samples exhibiting over 1,000 AU of activity were assigned “+++.” The data are reported in Tables 4A and 4B.

Table 4: Nuclear hormone receptors and helper genes strategies are assayed using R-SAT™

TABLE 4A

| | | SRC1/ | | ERK2/ |
|---|---|---|---|---|
| | | SEQ ID NOs: | DRIP205/SEQ | SEQ ID |
| Receptor/SEQ ID NO:* | | 145 and 147* | ID NO: 161* | NO: 213* |
| TR beta | 5 | − | ++ | ++ |
| RAR beta | 9 | ++ | ++ | ++ |
| PPARγ | 21 23, 25, 27 | +++ | + | +++ |
| FXR | 49 | +++ | +++ | +++ |
| LXR beta | 45 | +++ | − | ++ |
| VDR | 51 | ++ | + | + |
| PXR | 53, 55, 57 | ++ | − | + |
| RXR alpha | 69 | + | ++ | ++ |
| ER beta | 93 | +++ | ++ | +++ |
| AR | 107 | + | ++ | − |
| MR | 103 | +++ | ++ | + |
| GR | 101 | ++ | − | + |

TABLE 4B

| | | | | ERK2/ |
|---|---|---|---|---|
| Receptor/ | | SRC1/SEQ ID NOs: | DRIP205/SEQ | SEQ ID |
| SEQ ID NO:* | | 145, 147* | ID NO: 161* | NO: 213* |
| SHP | 143 | + | − | + |
| revErb alpha | 29 | − | − | + |
| ROR gamma | 43 | − | +++ | + |
| HNF4 alpha | 61 | +++ | ++ | +++ |
| TR2 alpha | 75 | + | − | − |
| TLX | 79 | − | − | + |
| COUP-TF beta | 87 | + | + | − |
| EAR2 | 89 | ++ | +++ | + |
| ERR gamma | 99 | − | − | ++ |
| NOR-1 | 123 | +++ | ++ | +++ |
| GCNF | 135 | + | − | + |

*The SEQ ID NOs: given are for nucleic acid receptors, helper genes and variants. The amino acid sequences are the next consecutive even number.

These data demonstrate that helper genes are often necessary to enable or improve the assays for nuclear hormone receptors.

### Example 8

### Selective Nuclear Receptor Modulators

This example shows how the disclosed methods can be used to screen for candidate molecules with activity against a particular receptor. Selective nuclear receptor modulators refer to a class of compounds with mixed agonist/antagonist characteristics. This specificity is cell-type dependent and has been associated with co-regulator recruitment in the case of estrogen modulators (Shang and Brown, 2002, Nature, 295:2465). More generally the design of selective nuclear receptor modulators is thought to provide the potential to identify novel drugs with a better therapeutic profile than those available currently. The amplification technology described herein and, for example, R-SAT™ allows for the distinction of a number of nuclear receptor-coregulator interactions (see Table 4). As such R-SAT™ is amenable to the identification of selective modulators of nuclear receptor activities.

### Example 9

### Importance of Kinases in the Nuclear Response

The following example demonstrates the importance of helper genes such as kinases in the pharmacological responses of nuclear receptors.

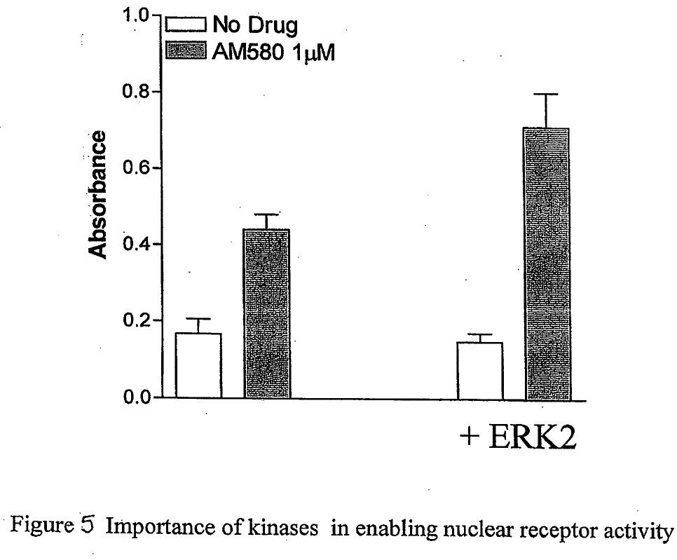

Cells were transfected with plasmid DNA encoding the nuclear receptor RARβ2 and β-Gal plasmid DNAs, as well as the MAP kinase ERK2. The transfected cells were contacted with the indicated amount of AM 590, a pan-retinoid agonist ligand. Cells were assayed using R-SAT™ as described in Example 1. The results are presented in FIG. 5, which represents a typical pharmacological profile of an agonist response of the retinoid receptor as determined by R-SAT. These data demonstrate that co-transfection of ERK2 improves the agonist response seen for RARβ2.

### Example 10

### Identification of Novel Interactions

This example demonstrates how the disclosed methods can be used to identify and dissect novel interactions between a particular receptor and signaling molecules.

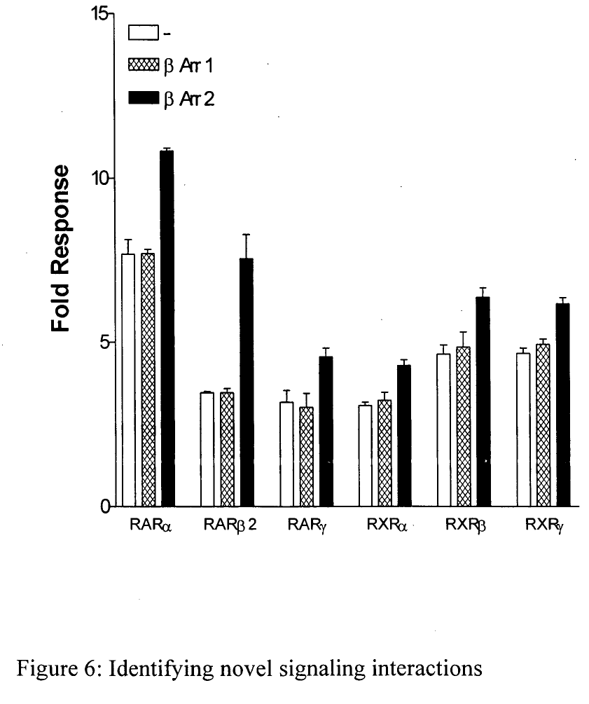

To determine whether β-arrestin 1 or β-arrestin 2, which are known to interact with a number of signaling molecules that link to MAPK signaling cascades, the ability of β-arrestin 1 or β-arrestin 2 to affect the activity of different retinoid nuclear receptors was assayed. Cells were co-transfected with the indicated RAR receptor, and either β-arrestin 1 or β-arrestin 2, as indicated, as well as the β-Gal plasmid DNA, as described in Example 1. The cells were contacted with AM 590, and assayed using R-SAT™ as described in Example 1. The data are presented in FIG. 6. As shown, the signaling intermediate β-arrestin 2 (GenBank NM004313, NM199004 m, the disclosures of which are herein incorporated by reference in their entirety) but not β-arrestin 1 (GenBank NM004041, NM020251, the disclosures of which are herein incorporated by reference in their entirety) can positively modulate the activity of the RAR receptors, such as RARβ2.

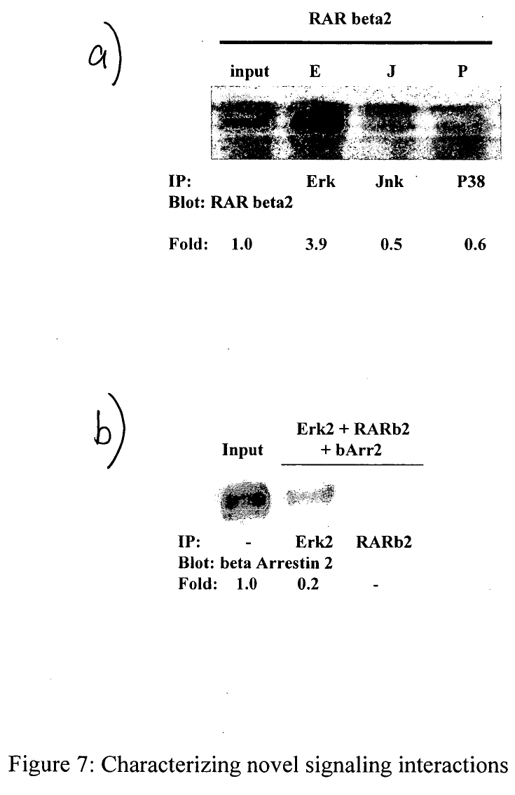

Co-immunoprecipitation experiments were used to confirm the interaction of β-arrestin 2 with RARβ2 and Erk. Cells co-transfected with plasmids encoding RARβ2 and Erk as described in Example 7 were scraped off of plates, spun down, and resuspended in lysis buffer (25 mM HEPES, 0.3M NaCl, 1.5 mM MgCl2, 0.2 mM EDTA and 0.5% Triton and a protease inhibitor cocktail. 50 μg of cell extracts were pre-cleared with pre-immune serum, incubated with protein A/G sepharose and an anti-Erk, Jnk, or P38 antibody (as indicated) for 2 hours, then washed extensively. Immune complexes were separated on denaturing polyacrylamide gels using SDS-PAGE and the proteins were blotted onto Immobilon-P membranes (Millipore, Billercia, Mass.). Western blotting was performed as described in Piu et al. (2002), using an anti-RARβ2 antibody. The data in FIG. 7A demonstrate the interaction between RARβ2 and Erk. In contrast, no interaction was seen with RARβ2 and Jnk or p38.

In the set of experiments depicted in FIG. 7B, cells were co-transfected with Erk2, RARβ2, and β arrestin 2. Co-immunoprecipitation was performed as described above, using an anti-Erk2 or anti-RARβ2 antibody, as indicated. Anti-β arrestin 2 antibody was used in the Western blots. As shown in FIG. 7B β-arrestin 2 physically interacts with the MAP kinase ERK2, which as shown in FIG. 7A binds to and activates RARβ2.

The data from this Example validate the use of the methods described herein to identify and characterize novel interactions between nuclear receptors and other signaling proteins.

The various methods and techniques described above provide a number of ways to carry out the invention. Of course, it is to be understood that not necessarily all objectives or advantages described can be achieved in accordance with any particular embodiment described herein. Thus, for example, those skilled in the art will recognize that the methods can be performed in a manner that achieves or optimizes one advantage or group of advantages as taught herein without necessarily achieving other objectives or advantages as can be taught or suggested herein.

Furthermore, the skilled artisan will recognize the interchangeability of various features from different embodiments. Similarly, the various features and steps discussed above, as well as other known equivalents for each such feature or step, can be mixed and matched by one of ordinary skill in this art to perform methods in accordance with principles described herein.

Although the invention has been disclosed in the context of certain embodiments and examples, it will be understood by those skilled in the art that the invention extends beyond the specifically disclosed embodiments to other alternative embodiments and/or uses and obvious modifications and equivalents thereof. Accordingly, the invention is not intended to be limited by the specific disclosures of preferred embodiments herein, but instead by reference to claims attached hereto.

What is claimed is:

1. A method of assessing the effect of a candidate compound on the activity of a nuclear receptor comprising:
obtaining a cell expressing one or more nuclear receptors and one or more helper proteins, wherein at least one of said nuclear receptor and said helper protein is expressed from a nucleic acid which has been introduced into said cell;
contacting said cell with said candidate compound; and
determining whether said candidate compound influences the activity of said nuclear hormone receptor.

2. The method of claim 1 wherein both said one or more nuclear receptor and said one or more helper protein are expressed from a nucleic acid which has been introduced into said cell.

3. The method of claim 1, wherein said one or more nuclear receptor and said one or more helper protein are expressed from the same nucleic acid which has been introduced into said cell.

4. The method of claim 1, wherein said one or more nuclear receptor is expressed from a first nucleic acid which has been introduced into said cell and said helper protein is expressed from a second nucleic acid which has been introduced into said cell.

5. The method of claim 1, wherein said determining step comprises comparing the activity of said nuclear hormone receptor in a first cell which expresses said nuclear receptor and said helper protein and which has been contacted with said candidate compound to the activity of said nuclear receptor in a second cell which expresses said nuclear receptor and said helper protein and which has not been contacted with said candidate compound, wherein said candidate compound is determined to influence the activity of said nuclear receptor if said activity of said nuclear receptor in said first cell is significantly different from the activity of said nuclear receptor in said second cell.

6. The method of claim 1, wherein said one or more nuclear receptors is encoded by a nucleic acid selected from the group consisting of SEQ ID NOs.: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, and 143 and said one or more helper proteins is encoded by a nucleic acid selected from the group consisting of 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, and 241.

7. The method of claim 1 wherein said one or more nuclear receptors is encoded by a nucleic acid comprising a nucleic acid having at least 70% nucleotide sequence identity to a nucleotide sequence selected from the group consisting of SEQ ID NOS.: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, and 143 and said one or more helper proteins is encoded by a nucleic acid comprising a nucleic acid having at least 70% nucleotide sequence identity to a nucleotide sequence selected from the group consisting of SEQ ID NOS.:145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, and 241.

8. The method of claim 1, wherein said one or more nuclear receptors comprises an amino acid sequence selected from the group consisting of SEQ ID NOs: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, and 144 and wherein said one or more helper proteins comprises an amino acid sequence selected from the group consisting of SEQ ID NOs: 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, and 242.

9. The method of claim 1, wherein said one or more nuclear receptors comprises an amino acid sequence having at least 70% amino acid identify to an amino acid sequence selected from the group consisting of SEQ ID NOs: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, and 144 and wherein said one or more helper proteins comprises an amino acid sequence having at least 70% amino acid identify to an amino acid sequence selected from the group consisting of SEQ ID NOs: 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, and 242.

10. The method of claim 1 wherein the combination of said nucleic acid sequence encoding said one or more nuclear receptors and said one or more helper proteins are selected from the group consisting of:
SEQ ID NO: 5 and SEQ ID NOs: 161 or 213;
SEQ ID NO: 9 and SEQ ID NOs: 145, 147, 161, or 213;
SEQ ID NOs: 21, 23, 25, or 27 and SEQ ID NOs: 145, 147, or 213;
SEQ ID NO: 49 and SEQ ID NOs: 145, 147, 161, or 213;
SEQ ID NO: 45 and SEQ ID NOs: 145 or 213;
SEQ ID NO: 51 and SEQ ID NOs: 145 or 147;
SEQ ID NOs: 53, 55, or 57 and SEQ ID NOs: 145 or 147;
SEQ ID NO: 69 and SEQ ID NOs: 161 or 213;
SEQ ID NO: 93 and SEQ ID NOs: 145, 147, 161, or 213;
SEQ ID NO: 107 and SEQ IDNO: 161;
SEQ ID NO: 103 and SEQ ID NOs: 145, 147, or 161;
SEQ ID NO: 101 and SEQ ID NOs: 145 or 147;
SEQ ID NO: 143 and SEQ ID NOs: 145, 147 or 161;
SEQ ID NO: 29 and SEQ ID NO: 213;
SEQ ID NO: 43 and SEQ ID NO: 161;
SEQ ID NO: 61 and SEQ ID NOs: 145, 147, 161 or 213;
SEQ ID NO: 75 and SEQ ID NOs: 145 or 147;
SEQ ID NO: 79 and SEQ ID NO: 161;
SEQ ID NO: 87 and SEQ ID NO: 145, 147, or 161;
SEQ ID NO: 89 and SEQ ID NOs: 145, 147 or 161;
SEQ ID NO: 99 and SEQ ID NO:161;
SEQ ID NOs: 123, 125, or 127 and SEQ ID NOs: 145, 147, 161, or 213; and
SEQ ID NO: 135 and SEQ ID NOs: 145, 147 or 213.

11. The method of claim 6 wherein the combination of the amino acid sequence of said one or more nuclear receptors and said one or more helper proteins are selected from the group consisting of:
SEQ ID NO: 6 and SEQ ID NOs: 162 or 214;
SEQ ID NO: 10 and SEQ ID NOs: 146, 148, 162, or 214;
SEQ ID NOs: 22, 24, 26 or 28 and SEQ ID NOs: 146, 148, or 214;
SEQ ID NO: 50 and SEQ ID NOs: 146, 148, 162, or 214;
SEQ ID NO: 46 and SEQ ID NOs: 146, 148 or 214;
SEQ ID NO: 52 and SEQ ID NOs: 146 or 148;
SEQ ID NOs: 54, 56, or 58 and SEQ ID NOs: 146 or 148;
SEQ ID NO: 70 and SEQ ID NOs: 162 or 214;
SEQ ID NO: 94 and SEQ ID NOs: 146, 148, 162, or 214;
SEQ ID NO: 108 and SEQ ID NO: 162;
SEQ ID NO: 104 and SEQ ID NOs: 146, 148, or 162;
SEQ ID NO: 102 and SEQ ID NOs: 146 or 148;
SEQ ID NO: 144 and SEQ ID NOs: 146, 148, or 162;
SEQ ID NO: 30 and SEQ ID NO: 214;
SEQ ID NO: 44 and SEQ ID NO: 162;
SEQ ID NO: 62 and SEQ ID NOs: 146, 148, 162, or 214;
SEQ ID NO: 76 and SEQ ID NOs: 146 or 148;
SEQ ID NO: 80 and SEQ ID NO: 162;
SEQ ID NO: 89 and SEQ ID NO: 146, 148, or 162;
SEQ ID NO: 90 and SEQ ID NOs: 146, 148, or 162;
SEQ ID NO: 100 and SEQ ID NO:162;
SEQ ID NOs: 124, 126, or 128 and SEQ ID NOs: 146, 148, 162, or 214; and
SEQ ID NO: 136 and SEQ ID NOs: 146, 148 or 214.

12. The method of claim 1, wherein the combination of the nuclear receptor expressed by said cell and the helper protein expressed by said cell are selected from the group consisting of:
TR beta and DRIP 205 or ERK2;
RAR beta and SRC1, DRIP205 or ERK2;
PPAR gamma and SRC1 or ERK2;
FXR and SRC1, DRIP205 or ERK2;
LXR beta and SRC1 or ERK2;
VDR and SRC1;
PXR and SRC1;
RXR alpha and DRIP205 or ERK2;
ER beta and SRC1, DRIP205 or ERK2;
AR and DRIP205;
MR and SRC1 or DRIP205;
GR and SRC 1;
SHP and SRC1 or ERK2;
RevERb alpha and ERK2;
ROR gamma and DRIP205;
HNF4 alpha and SRC1, DRIP205 or ERK2;
TR2 alpha and SRC1;
TLX and ERK2;
COUP-TF beta and SRC1 or DRIP205;
EAR2 and SRC1, DRIP205 or ERK2;
ERR gamma and ERK2;
NOR-1 and SRC1, DRIP205 or ERK2; and
GCNF and SRC1 or ERK2.

13. The method of claim 1, wherein said determining step comprises determining whether said compound influences the activity of said one or more nuclear receptors by evaluating a cellular parameter selected from the group consisting of morphology, phosphorylation, differentiation, apoptosis, process formation, motility, gene expression, expression of a cellular receptor, and a phenotypic change

14. The method of claim 1, further comprising introducing a nucleic acid comprising a promoter from which the level of transcription is responsive to activation of said nuclear receptor into said cell, said promoter being operably linked to a nucleic acid encoding a detectable product and determining whether said candidate compound influences the activity of said nuclear receptor by measuring the amount of said detectable product.

15. A method of identifying interaction between a nuclear receptor and one or more helper proteins, said method comprising:
co-transfecting a first cell culture with DNA encoding a nuclear receptor and DNA encoding a marker of cell amplification, along with DNA encoding one or more helper proteins;
incubating the cell culture with varying concentrations of a ligand which is an agonist or antagonist for said nuclear receptor for a period of time sufficient to permit cell amplification of said transfected cells in said first cell culture;
co-transfecting a second cell culture with DNA encoding a nuclear receptor and DNA encoding a marker of cell amplification;
incubating the cell culture with varying concentrations of a ligand which is an agonist or antagonist for said nuclear receptor for a period of time sufficient to permit cell amplification of said transfected cells in said second cell culture;
determining whether said one or more helper proteins interact with said nuclear receptor by comparing the level of amplification of transfected cells expressing said nuclear receptor and said one or more helper proteins to the level of amplification of cells which were transfected with DNA encoding said nuclear receptor but which were not transfected with DNA encoding said one or more helper proteins.

16. A method according to claim 15, wherein said one or more helper proteins is a coactivator.

17. A method according to claim 15, wherein said one or more helper proteins is a corepressor.

18. A method according to claim 15, wherein said one or more helper proteins is a kinase.

19. A method according to claim 15, wherein said one or more helper proteins is a signaling molecule.

20. A method according to claim 15, wherein said one or more helper proteins comprises at least two helper proteins selected from the group consisting of corepressors, and kinases, signaling molecules.

21. The method of claim 15, wherein said DNA encoding said nuclear receptor comprises a sequence selected from the group consisting of SEQ ID NOs. 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, and 143.

22. The method of claim 15, wherein said DNA encoding said nuclear receptor encodes a polypeptide comprising a sequence selected from the group consisting of SEQ ID NOs.: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, and 144.

23. The method of claim 15, wherein said DNA encoding said one or more helper proteins comprises a sequence selected from the group consisting of SEQ ID NOs.: 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, and 241.

24. The method of claim 15, wherein said DNA encoding said one or more helper proteins encodes a polypeptide comprising a sequence selected from the group consisting of SEQ ID NOs.: 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, and 242.

25. The method of claim 15, wherein said DNA encoding said nuclear receptor and said DNA encoding said marker of cell amplification are on the same vector.

26. The method of claim 15, wherein said DNA encoding said nuclear receptor and said DNA encoding said marker of cell amplification are on separate vectors.

27. A method of identifying a substance which is a ligand of a nuclear receptor, said method comprising:
incubating a cell culture which comprises a mixture of cells transfected with DNA encoding a nuclear receptor, DNA encoding a marker of cell amplification and DNA encoding one or more helper proteins and untransfected cells, with a test substance which is a potential agonist or antagonist for said nuclear receptor for a period of time sufficient to permit cell amplification of said transfected cells; and
determining any increase or decrease in cell amplification by measuring the level of the marker in said transfected cells.

28. A method of identifying a substance which is a selective modulator of a particular combination of a nuclear receptor and one or more helper proteins, said method comprising:
co-transfecting a first cell culture comprising cells of a first cell type with DNA encoding a nuclear receptor and DNA encoding a marker of cell amplification, along with DNA encoding said one or more helper proteins;
incubating the first cell culture with a test substance;
determining whether said test substance increases or decreases amplification of said transfected cells of said first cell type relative to untransfected cells of said first cell type;
co-transfecting a second cell culture comprising cells of a second cell type with DNA encoding said nuclear receptor and DNA encoding said marker of cell amplification, along with DNA encoding said one or more helper proteins;
incubating the second cell culture with said test substance;
determining whether said test substance increases or decreases amplification of said transfected cells of said second cell type relative to untransfected cells of said second cell type;
wherein said test substance is a selective modulator of said nuclear receptor if the effects of said test substance on said first cell type are opposite to the effects of said test substance on said second cell type.

29. A method of identifying a substance which is a selective modulator of a particular combination of a nuclear receptor and one or more helper proteins, said method comprising:
co-transfecting a first cell culture with DNA encoding a nuclear receptor and DNA encoding a marker of cell amplification, along with DNA encoding one or more first helper proteins;
incubating the first cell culture with a test substance;
determining whether said test substance increases or decreases amplification of said transfected cells in said first cell culture relative to untransfected cells;
co-transfecting a second cell culture with DNA encoding said nuclear receptor and DNA encoding a marker of cell amplification, along with DNA encoding a second one or more helper proteins, wherein said second one or more helper proteins are distinct from said first one or more helper proteins;
incubating the second cell culture with said test substance;
determining whether said test substance increases or decreases amplification of said transfected cells in said second cell culture relative to untransfected cells;
wherein said test substance is a selective modulator of said nuclear receptor if the effects of said test substance on said first cell culture are opposite to the effects of said test substance on said second cell culture.

30. A method of identifying a substance which is a selective modulator of a nuclear receptor comprising:
co-transfecting a first cell culture comprising cells of a first cell type with DNA encoding a nuclear receptor and DNA encoding a marker of cell amplification;
incubating the first cell culture with a test substance;
determining whether said test substance increases or decreases amplification of said transfected cells of said first cell type relative to untransfected cells of said first cell type;
co-transfecting a second cell culture comprising cells of a second cell type with DNA encoding said nuclear receptor and DNA encoding said marker of cell amplification;
incubating the second cell culture with said test substance;
determining whether said test substance increases or decreases amplification of said transfected cells of said second cell type relative to untransfected cells of said second cell type;
wherein said test substance is a selective modulator of said nuclear receptor if the effects of said test substance on said first cell type are opposite to the effects of said test substance on said second cell type.

31. A method of detecting a substance which is a ligand of two nuclear receptors comprising:
incubating a cell culture which comprises a mixture of cells transfected with DNA encoding a first nuclear receptor, DNA encoding a second nuclear receptor, and DNA encoding a marker of cell amplification with a test substance which is a potential agonist or antagonist for said nuclear receptor for a period of time sufficient to permit cell amplification of said transfected cells; and
determining any increase or decrease in cell amplification by measuring the level of the marker of cell amplification in said transfected cells.

32. A method of detecting a substance which is a selective modulator of a particular combination of two nuclear receptors and one or more helper proteins comprising:
co-transfecting a first cell culture comprising cells of a first cell type with DNA encoding a first nuclear receptor, DNA encoding a second nuclear receptor, DNA encoding one or more helper proteins, and DNA encoding a marker of cell amplification;
incubating the first cell culture with a test substance;
determining whether said test substance increases or decreases amplification of said transfected cells in said first cell culture relative to untransfected cells in said first cell culture;
co-transfecting a second cell culture comprising cells of a second cell type with DNA encoding said first nuclear receptor, DNA encoding said second nuclear receptor and DNA encoding a marker of cell amplification;
incubating the second cell culture with said test substance; and
determining whether said test substance increases or decreases amplification of said transfected cells of said second cell type relative to untransfected cells of said second cell type;
wherein said test substance is a selective modulator of said nuclear receptor if the effects of said test substance on said first cell type are opposite to the effects of said test substance on said second cell type.

33. A method of identifying a substance which is a selective modulator of a particular combination of two nuclear receptors and one or more helper proteins comprising:
co-transfecting a first cell culture with DNA encoding a first nuclear receptor, DNA encoding a second nuclear receptor and DNA encoding a marker of cell amplification, along with DNA encoding one or more first helper proteins;
incubating the first cell culture with a test substance;
determining whether said test substance increases or decreases amplification of said transfected cells in said first cell culture relative to untransfected cells;
co-transfecting a second cell culture with DNA encoding said first nuclear receptor, DNA encoding said second nuclear receptor and DNA encoding a marker of cell amplification, along with DNA encoding a second one or more helper proteins, wherein said second one or more helper proteins are distinct from said first one or more helper proteins;
incubating the second cell culture with said test substance;
determining whether said test substance increases or decreases amplification of said transfected cells in said second cell culture relative to untransfected cells;
wherein said test substance is a selective modulator of said nuclear receptor if the effects of said test substance on said first cell culture are opposite to the effects of said test substance on said second cell culture.
